(19) United States
(12) Patent Application Publication
(10) Pub. No.: US 2022/0313958 A1
(43) Pub. Date: Oct. 6, 2022

(54) Instrument Delivery Devices, Systems, and Methods

(71) Applicant: Becton, Dickinson and Company, Franklin Lakes, NJ (US)

(72) Inventors: Weston F. Harding, Lehi, UT (US); Bart D. Peterson, Farmington, UT (US); Bradley M. Wilkinson, North Haledon, NJ (US); Curtis H. Blanchard, Riverton, UT (US); Megan Scherich, Salt Lake City, UT (US)

(21) Appl. No.: 17/709,935

(22) Filed: Mar. 31, 2022

Related U.S. Application Data
(60) Provisional application No. 63/170,381, filed on Apr. 2, 2021.

(51) Int. Cl. A61M 25/06; A61M 25/09; A61M 25/01

(52) U.S. Cl. CPC A61M 25/0606; A61M 25/09041; A61M 25/0113

(57) ABSTRACT

An instrument delivery device may include a housing, which may include a distal end and a proximal end. The distal end may be configured to couple to an intravenous catheter device. The proximal end may include a proximal connector. The proximal connector may be adjustable between a first position with respect to the housing to a second position with respect to the housing. An instrument may be disposed within the housing. An advancement wheel may extend out from the housing. In response to the advancement wheel being rotated, the instrument may be advanced through the distal end of the housing. A housing of another instrument delivery device may include an advancement element, which may include a first push tab and/or a second push tab.

### CROSS-REFERENCE TO RELATED APPLICATION

The present application claims priority to U.S. Provisional Application No. 63/170,381, entitled “Instrument Delivery Devices, Systems, and Methods”, filed Apr. 2, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of intravenous (IV) catheter device includes a catheter that is over-the-needle. As its name implies, the catheter that is over-the-needle may be mounted over an introducer needle having a sharp distal tip. The IV catheter device may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to instrument delivery devices to facilitate advancement and/or retraction of an instrument within an IV catheter, as well as related systems and methods. In some embodiments, the instrument delivery device may include a housing, which may include a distal end and a proximal end. In some embodiments, the distal end of the housing may be configured to couple to an intravenous (IV) catheter device. As used in the present disclosure, the term “instrument” may include a guidewire, a probe, a guidewire or a probe with one or more sensors, or another suitable instrument.

In some embodiments, an inner surface of the housing may include a stop member. In some embodiments, the instrument delivery device may include a spool disposed within the housing and an instrument wound around the spool. In some embodiments, the instrument delivery device may include an advancement wheel. In some embodiments, the advancement wheel may extend out from the housing. In some embodiments, in response to the advancement wheel being rotated, the spool may rotate to cause the instrument to advance through the distal end of the housing. In some embodiments, an outer surface of the advancement wheel may include another stop member configured to contact the stop member and stop rotation of the advancement wheel more than a full turn.

In some embodiments, the stop member and/or the other stop member may include a protrusion. In some embodiments, the housing may include a fluid pathway extending through the distal end and the proximal end of the housing. In some embodiments, the housing may include an instrument channel that extends from the spool to the fluid pathway. In some embodiments, the instrument delivery device may include a seal that isolates the instrument channel from the fluid pathway, and the instrument may extend through the seal. In some embodiments, the proximal end of the housing may include a luer connector or another suitable type of connector.

In some embodiments, an instrument delivery device may include a housing, which may include a distal end and a proximal end. In some embodiments, the distal end of the housing may be configured to couple to an intravenous catheter device. In some embodiments, an inner surface of the housing may include a housing stop member.

In some embodiments, the instrument delivery device may include a first wheel. In some embodiments, an inner surface of the first wheel may include a first wheel stop. In some embodiments, the instrument delivery device may include a second wheel, which may include a tab. In some embodiments, in response to the instrument delivery device being disposed in a first configuration, the first wheel and/or the second wheel may be prevented from rotating in a first direction. In some embodiments, in response to the instrument delivery device being disposed in a first configuration, the first wheel and/or the second wheel may be configured to rotate in a second direction opposite the first direction. In some embodiments, the first wheel may be configured to rotate more than one full turn in the second direction.

In some embodiments, a gap may be disposed between the housing stop and the first wheel stop. In some embodiments, in response to the instrument delivery device being disposed in a first configuration, the tab may bridge the gap between the housing stop and the first wheel stop. In some embodiments, in response to the instrument delivery device being disposed in the first configuration, the tab may be disposed between the housing stop member and the first wheel stop member and may contact the housing stop member and the first wheel stop member. In some embodiments, in response to the instrument delivery device being disposed in the first configuration, the first wheel may be configured to rotate independent from the housing and the second wheel in the second direction until the first wheel stop member contacts the tab.

In some embodiments, in response to the first wheel rotating independent from the housing and the second wheel in the second direction until the first wheel stop member contacts the tab, the first wheel and the second wheel are configured to rotate together further in the second direction until the instrument delivery device is disposed in a second configuration. In some embodiments, in the second configuration, the tab may be disposed between the housing stop member and the first wheel stop member and may contact the housing stop member and the first wheel stop member.

In some embodiments, the instrument delivery device may include an instrument. In some embodiments, the instrument may be in a fully retracted position in response to the instrument delivery device being in the first configuration. In some embodiments, the instrument may be in a fully advanced position in response to the instrument delivery device being in the second configuration. In some embodiments, the first wheel may be configured to rotate in the second direction to advance the instrument in a distal direction through the distal end of the housing.

In some embodiments, an instrument delivery device may include a housing, which may include a distal end and a proximal end. In some embodiments, the distal end may be configured to couple to an IV catheter device. In some embodiments, an inner surface of the housing may include a housing stop member and a housing detent. In some embodiments, the instrument delivery device may include an axle. In some embodiments, the instrument delivery device may include a first wheel configured to rotate with the axle. In some embodiments, an inner surface of the first wheel may include a first wheel stop member and a first wheel detent.

In some embodiments, the instrument delivery device may include a second wheel disposed on the axle and configured to rotate with the axle and move axially along the axle. In some embodiments, the second wheel may include a tab. In some embodiments, in response to the instrument delivery device being in a first configuration, the tab may be disposed within the housing detent and the first wheel detent. In these embodiments, the first wheel may be prevented from rotating in a first direction but may be configured to rotate in a second direction opposite the first direction. In some embodiments, the first wheel may be configured to rotate more than one full turn in the second direction.

In some embodiments, the instrument delivery device may include an instrument. In some embodiments, the first wheel may be configured to rotate in the second direction to advance the instrument in a distal direction through the distal end of the housing. In some embodiments, in response to the first wheel rotating in the second direction from the first configuration, the tab may be removed from the first wheel detent before the tab is removed from the housing detent. In some embodiments, the tab may be removed from the housing detent in response to the tab sliding toward the inner surface of the first wheel.

In some embodiments, the inner surface of the housing may include another housing detent. In some embodiments, the inner surface of the first wheel may include a first wheel ramped surface. In some embodiments, the first wheel detent may be disposed between the first wheel stop member and the first wheel ramped surface. In some embodiments, the first wheel ramped surface may be ramped towards the first wheel detent.

In some embodiments, the first wheel may be configured to rotate from the first configuration to a second configuration. In some embodiments, the tab may be disposed within the other housing detent and the first wheel detent in the second configuration. In some embodiments, in order to move from the first configuration to the second configuration, the tab may contact the housing stop member. In some embodiments, in response to the tab contacting the housing stop member and further rotation of the first wheel in the second direction, the tab may move along the first wheel ramped surface and may be pushed by the first wheel ramped surface towards the inner surface of the housing and into the other housing detent.

In some embodiments, the inner surface of the housing may include a housing ramped surface and another housing ramped surface. In some embodiments, the housing detent may be disposed between the housing stop member and the housing ramped surface. In some embodiments, the other housing detent may be disposed between the housing stop member and the other ramped surface and on an opposite side of the housing stop member as the housing detent.

In some embodiments, the housing ramped surface may be ramped towards the housing detent. In some embodiments, the first wheel ramped surface may be ramped towards the first wheel detent. In some embodiments, the housing ramped surface and the first wheel ramped surface may be ramped in differing or opposing directions. In some embodiments, the housing detent may be disposed across from the first wheel detent in the first configuration.

In some embodiments, an instrument delivery device may include a housing, which may include a distal end and a proximal end. In some embodiments, the distal end of the housing may be configured to couple to an intravenous catheter device. In some embodiments, the proximal end of the housing may include a proximal connector. In some embodiments, the proximal connector may be adjustable between a first position with respect to the housing to a second position with respect to the housing. In some embodiments, the instrument may be disposed within the housing. In some embodiments, the instrument delivery device may include an advancement wheel, which may extend out from the housing. In some embodiments, in response to the advancement wheel being rotated, the instrument may be advanced through the distal end of the housing.

In some embodiments, the proximal connector may include a luer connector. In some embodiments, the proximal connector may be pivotally coupled to the housing and configured to move along an axis between the first position and the second position. In some embodiments, the proximal connector may include a first detent and/or a second detent. In some embodiments, an inner surface of the housing may include a protrusion. In some embodiments, in response to the proximal connector being in the first position, the protrusion may be disposed within the first detent. In some embodiments, in response to the proximal connector being in the second position, the protrusion may be disposed within the second detent.

In some embodiments, the first position may be a horizontal position with respect to the housing. In some embodiments, the second position may be a downwardly inclined position with respect to the housing. In some embodiments, in response to the proximal connector being in the horizontal position, the protrusion may be disposed within the first detent. In some embodiments, in response to the proximal connector being in the downwardly inclined position, the protrusion may be disposed within the second detent.

In some embodiments, the distal end of the housing may include a distal connector. In some embodiments, the distal connector may include a blunt cannula and two lever arms disposed on opposite sides of the blunt cannula. In some embodiments, the blunt cannula may include a luer shape. In some embodiments, a proximal end of each of the two lever arms may include a stop protrusion.

In some embodiments, an instrument delivery device may include a housing, which may include a proximal end, a distal end, and a slot. In some embodiments, an inner surface of the housing may include a groove disposed within the housing between the proximal end of the housing and the distal end of the housing. In some embodiments, the instrument delivery device may include an advancement element extending through the slot and configured to move linearly along the slot between a retracted position and an advanced position. In some embodiments, the advancement element may include a first push tab and/or a second push tab. In some embodiments, an instrument may include a first end and a second end. In some embodiments, in response to movement of the advancement element from the retracted position to the advanced position, the second end of the instrument may be advanced beyond the distal end of the housing.

In some embodiments, the advancement element may include an arc-shaped channel. In some embodiments, the instrument may extend through the arc-shaped channel. In some embodiments, the first end of the instrument may be fixed. In some embodiments, in response to movement of the advancement element a first distance, the second end of the instrument may be configured to advance distally a second distance. In some embodiments, the second distance may be at least twice the first distance. In some embodiments, an inner surface of the housing may include a first groove and/or a second groove. In some embodiments, the second groove may be generally parallel to the first groove. In some embodiments, the instrument may be disposed within the first groove and/or the second groove.

In some embodiments, the instrument may be oriented in a horizontal plane. In some embodiments, a width of the housing may be greater than a height of the housing. In some embodiments, the first push tab may be disposed at a distal end of the advancement element. In some embodiments, the second push tab may be disposed at a proximal end of the advancement element and is aligned with the first push tab. In some embodiments, a height of the first push tab may be greater than a height of the second push tab.

In some embodiments, the housing may include a body and a tunnel disposed underneath the body. In some embodiments, the instrument delivery device may include a proximal connector and an extension tube extending through the tunnel and coupled to the proximal connector. In some embodiments, the body may extend proximal to the tunnel. In some embodiments, the body may limit upward movement of the proximal connector.

In some embodiments, a bottom surface of the body may include an upper flange, which may be arched or trapezoidal. In some embodiments, the body and/or the upper flange may include a corresponding shape to the proximal connector. In some embodiments, the upper flange may limit upward movement of the proximal connector.

In some embodiments, the bottom surface of the body may include a finger catch proximal to the upper flange. In some embodiments, the housing may include a lower flange extending distally from the finger catch and configured to limit side-to-side movement of the proximal connector. In some embodiments, the instrument may include a guidewire or another suitable instrument.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1 is a cross-sectional side view of an instrument delivery device, according to some embodiments;

FIG. 1A is an exploded rear view of an example instrument advancement mechanism of the instrument delivery device of FIG. 1;

FIG. 2 is a cross-sectional side view of another instrument delivery device, according to some embodiments;

FIG. 2A is a rear view of an instrument advancement mechanism of the instrument delivery device of FIG. 2;

FIG. 3 is a top perspective view of another instrument delivery device, according to some embodiments;

FIG. 4 is a cross-sectional side view of another instrument delivery device, according to some embodiments;

FIG. 5 is a cross-sectional side view of another instrument delivery device, according to some embodiments;

FIG. 6 is a cross-sectional side view of another instrument delivery device, according to some embodiments;

FIG. 7 is a cross-sectional side view of another instrument delivery device, according to some embodiments;

FIG. 8 is a cross-sectional side view of another instrument delivery device, according to some embodiments;

FIG. 9 is a cross-sectional side view of another instrument delivery device, according to some embodiments;

FIG. 10A is a cross-sectional side view of another instrument delivery device, illustrating the instrument delivery device in a first configuration, according to some embodiments;

FIG. 10B is a cross-sectional side view of the instrument delivery device of FIG. 10A, illustrating the instrument delivery device in a second configuration, according to some embodiments;

FIG. 10C is an upper perspective view of the instrument delivery device of FIG. 10A, illustrating the instrument delivery device in the first configuration, according to some embodiments;

FIG. 10D is an exploded rear view of an example instrument advancement mechanism of the instrument delivery device of FIG. 10A, according to some embodiments;

FIG. 11A is a cross-sectional side view of another instrument delivery device, illustrating the instrument delivery device in a first configuration, according to some embodiments;

FIG. 11B is a cross-sectional side view of the instrument delivery device of FIG. 11A, illustrating an example first wheel rotated independently from an example second wheel in a second direction from the first configuration, according to some embodiments;

FIG. 11C is a cross-sectional side view of the instrument delivery device of FIG. 11A, illustrating the first wheel rotated further in the second direction from the position of FIG. 11B, according to some embodiments;

FIG. 11D is a cross-sectional side view of the instrument delivery device of FIG. 11A, illustrating the first wheel and the second wheel rotated together in the second direction from the position of FIG. 11C, according to some embodiments;

FIG. 11E is a cross-sectional side view of another instrument delivery device, illustrating the instrument delivery device in a second configuration, according to some embodiments;

FIG. 12A is a cross-sectional front view of another instrument delivery device, illustrating the instrument delivery device in a first configuration, according to some embodiments;

FIG. 12B is a cross-sectional front view of the instrument delivery device of FIG. 12A, illustrating the instrument delivery device in the first configuration, according to some embodiments;

FIG. 12C is a cross-sectional side view of the instrument delivery device of FIG. 11A, illustrating an example first wheel rotated independently from an example second wheel in a second direction from the first configuration, according to some embodiments;

FIG. 12D is a cross-sectional front view of the instrument delivery device of FIG. 12A, illustrating the first wheel rotated independently from the second wheel in the second direction from the first configuration, according to some embodiments;

FIG. 12E is a cross-sectional side view of the instrument delivery device of FIG. 11A, illustrating an example tab moved towards the first wheel, according to some embodiments;

FIG. 12F is a cross-sectional front view of the instrument delivery device of FIG. 12A, illustrating the tab moved towards the first wheel, according to some embodiments;

FIG. 12G is a cross-sectional side view of the instrument delivery device of FIG. 12A, illustrating the tab and the first wheel rotated in the second direction from the position of FIG. 12E-12F, according to some embodiments;

FIG. 12H is a cross-sectional side view of the instrument delivery device of FIG. 12A, illustrating the tab and the first wheel rotated further in the second direction from the position of FIG. 12G, according to some embodiments;

FIG. 12I is a cross-sectional front view of the instrument delivery device of FIG. 12A, illustrating the tab and the first wheel rotated further in the second direction from the position of FIG. 12G, according to some embodiments;

FIG. 12J is a cross-sectional side view of the instrument delivery device of FIG. 12A, illustrating the instrument delivery device in a second configuration, according to some embodiments;

FIG. 12K is a cross-sectional front view of the instrument delivery device of FIG. 12A, illustrating the instrument delivery device in a second configuration, according to some embodiments;

FIG. 13A is a side view of another example instrument delivery device, illustrating an example proximal connector, according to some embodiments;

FIG. 13B is a side view of the instrument delivery device of FIG. 13A, illustrating an example blood collection device coupled to the proximal connector, according to some embodiments;

FIG. 13C is a side view of the instrument delivery device of FIG. 13A, illustrating the blood collection device and the proximal connector in a downwardly inclined position with respect to an example housing, according to some embodiments;

FIG. 13D is a cross-sectional view of a portion of the instrument delivery device of FIG. 13A, according to some embodiments;

FIG. 14A is an upper perspective view of an example distal connector, according to some embodiments;

FIG. 14B is an upper perspective view of the distal connector, according to some embodiments;

FIG. 15A is an upper perspective view of another example instrument delivery device, according to some embodiments;

FIG. 15B is a longitudinal cross-sectional view of the instrument delivery device of FIG. 15A;

FIG. 15C is a cross-sectional view of the instrument delivery device of FIG. 15A along the line 15C-15C of FIG. 15A, according to some embodiments;

FIG. 15D is an enlarged view of a portion of the instrument delivery device of FIG. 15C, according to some embodiments;

FIG. 15E is a cross-sectional view of the instrument delivery device of FIG. 15A along the line 15E-15E of FIG. 15A, according to some embodiments;

FIG. 16A is an upper perspective view of an example instrument delivery device, according to some embodiments;

FIG. 16B is an enlarged upper perspective view of a portion of the instrument delivery device of FIG. 16A, according to some embodiments;

FIG. 16C is a cross-sectional view of the instrument delivery device of FIG. 16A along the line 16C-16C of FIG. 16A, with an example advancement element removed for illustrative purposes, according to some embodiments;

FIG. 16D is a cross-sectional view of the instrument delivery device of FIG. 16A along the line 16D-16D of FIG. 16A, according to some embodiments;

FIG. 16E is an upper perspective view of the instrument delivery device of FIG. 16A, according to some embodiments;

FIG. 16F is a lower perspective view of the instrument delivery device of FIG. 16A, illustrating an example proximal connector in a horizontal position, according to some embodiments;

FIG. 16G is an enlarged lower perspective view of a portion of the instrument delivery device of FIG. 16A, according to some embodiments;

FIG. 16H is an enlarged lower perspective view of an example finger catch and an example lower flange extending therefrom, according to some embodiments;

FIG. 16I is an enlarged lower perspective view of the finger catch of FIG. 16H, illustrating the proximal connector in a downwardly inclined position, according to some embodiments;

FIG. 16J is an enlarged cross-sectional view through an example distal end of the proximal connector and an example upper flange, according to some embodiments;

FIG. 16K is an enlarged cross-sectional view along a longitudinal axis of the proximal connector, according to some embodiments;

FIG. 16L is an enlarged lower perspective view of another example upper flange, according to some embodiments; and

FIG. 16M is a cross-sectional view through an example distal end of the proximal connector and the upper flange of FIG. 16L, according to some embodiments.

### DETAILED DESCRIPTION OF THE EMBODIMENTS

In the specification and the claims, the term “IV catheter device” should be construed as any device that includes an intravenous (“IV”) catheter. The term “instrument delivery device” should be construed as any device that is configured to advance and/or retract an instrument within an IV catheter. In some embodiments, an instrument delivery device may be a separate device from an IV catheter device with which the instrument delivery device may be used. In other embodiments, an instrument delivery device may be in the form of an IV catheter device. In other words, an instrument delivery device may include an IV catheter in some embodiments. The term “instrument delivery mechanism” will be used to represent a variety of mechanisms and/or configurations of an instrument delivery device that facilitate advancement and/or retraction of an instrument within an IV catheter in accordance with embodiments of the present disclosure.

Prior to describing various examples of an instrument delivery device, general characteristics of some embodiments of an instrument delivery device will be described. An instrument delivery device includes a distal end, which will be oriented towards vasculature of a patient during use, and a proximal end opposite the distal end. In some embodiments, the distal end of the instrument delivery device may be configured to connect to an IV catheter device. In other embodiments, the distal end may include an IV catheter. In some embodiments, the proximal end of the instrument delivery device may be configured to allow a separate device to be connected to the instrument delivery device. For example, the proximal end may include an access port or a vacuum tube receiver that may form part of a fluid pathway that extends to the distal end of the instrument delivery device. In other embodiments, the distal end or another portion of the instrument delivery device may be configured to allow a separate device to be connected to the instrument delivery device. However, in some embodiments, the instrument delivery device may not be configured to enable a separate device to be connected to the instrument delivery device. For example, an instrument delivery device may be configured to deliver an instrument while not being configured for injecting fluid or withdrawing blood.

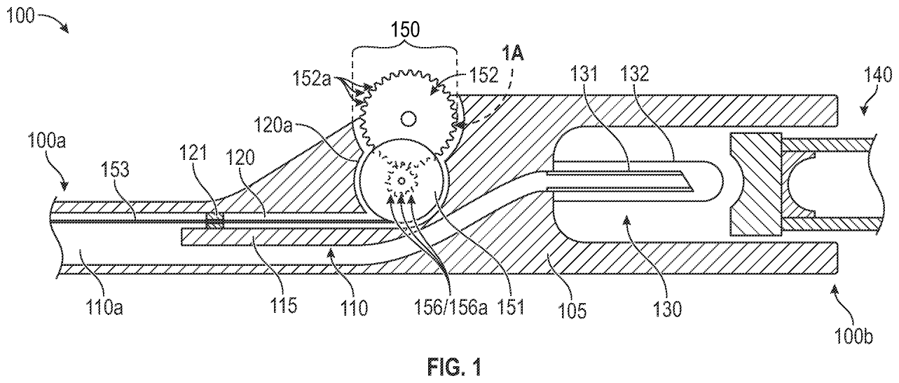

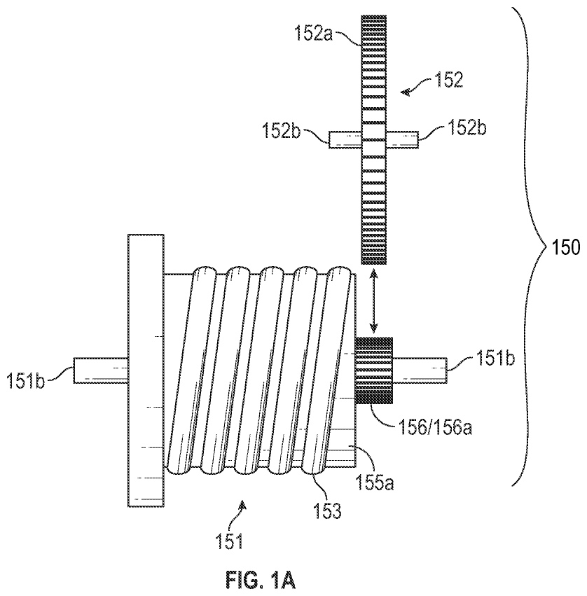

FIG. 1 illustrates an example of an instrument delivery device 100 that is configured in accordance with some embodiments of the present disclosure. In some embodiments, the instrument delivery device 100 may include a housing 105 having a distal end 100a and a proximal end 100b. In some embodiments, although only a portion of the distal end 100a is illustrated, as noted above, the distal end 100a could include any type of connector to enable the instrument delivery device 100 to be connected to an IV catheter device or could incorporate an IV catheter. In some embodiments, the proximal end 100b may be configured to form a vacuum tube receiver 130 having a needle 131 covered by a protective sheath 132.

In some embodiments, a fluid pathway 110 may extend within the instrument delivery device 100 from the needle 131 to the distal end 100a. Accordingly, when a vacuum tube 140 is inserted into the vacuum tube receiver 130, a blood sample can be collected through the fluid pathway 110. In some embodiments, the proximal end 100b may include a Luer connector or any other type of connector that is coupled to the fluid pathway 110.

In some embodiments, the instrument delivery device 100 may include an instrument delivery mechanism 150 that enables an instrument 153 to be advanced in a distal direction through an IV catheter and subsequently withdrawn in a proximal direction. In some embodiments, the instrument 153 may include a wire constructed of nickel titanium or another suitable material. In some embodiments, a compartment 120 may be formed within the instrument delivery device 100 and may house the instrument delivery mechanism 150. In some embodiments, a dividing wall 115 may create an instrument channel 121 that extends distally from the compartment 120 and joins the fluid pathway 110 at a distal portion 110a of the fluid pathway 110.

In some embodiments, to isolate the compartment 120 from the fluid pathway 110, a seal 122 (e.g., an elastomeric septum) may be positioned within and span the instrument channel 121. In some embodiments, the instrument 153 may extend through a slit or other opening formed within seal 122. In some embodiments, the seal 122 may provide support to instrument 153 to prevent it from buckling as it is advanced. Although the instrument channel 121 is illustrated as being substantially wider than instrument 153, in some embodiments, dimensions of at least a portion of the instrument channel 121 may be only slightly greater than the instrument 153 so that instrument channel 121 may provide support to prevent buckling of the instrument 153.

In some embodiments, the instrument delivery mechanism 150 may include a spool 155 and an advancement wheel 152, both of which may be configured to rotate within the compartment 120. In some embodiments, the spool 155 may be positioned adjacent to the advancement wheel 152 (i.e., towards the instrument channel 121 relative to the advancement wheel 152). In some embodiments, the advancement wheel 152 may be positioned to extend partially out from the compartment 120 to thereby enable a clinician to use his or her thumb or finger to rotate the advancement wheel 152. In some embodiments, the spool 155 may include a gear 156 having teeth 156a. Likewise, in some embodiments, the advancement wheel 152 may include teeth 152a and may therefore function as a gear. In some embodiments, the teeth 152a may interface with the teeth 156a so that the spool 155 is rotated when the advancement wheel 152 is rotated. In some embodiments, the teeth 152a are formed along the outermost edge of the advancement wheel 152. In other embodiments, however, teeth 152a may be formed along a portion of advancement wheel that is inset relative to the outermost edge.

FIG. 1A provides an exploded rear view of instrument delivery mechanism 150 in isolation, according to some embodiments. In some embodiments, the spool 155 and the advancement wheel 152 may include axles 155b and 152b, respectively, by which these components are positioned within the compartment 120 and around which these components rotate. In some embodiments, the spool 155 may include a spool drum 155a around which the instrument 153 may be wound. Therefore, when the spool 155 is rotated, the rotation may cause the instrument 153 to be advanced or retracted along the instrument channel 121 depending on the direction in which the advancement wheel 152 is rotated. In some embodiments, the gear formed by the advancement wheel 152 may have a larger diameter than the gear 156 to thereby cause the instrument 153 to be advanced or retracted a larger distance relative to the amount of rotation of the advancement wheel 152. In contrast, in other embodiments, the gear formed by the advancement wheel 152 may have an equal or smaller diameter than the gear 156. In such embodiments, the instrument 153 may advance or retract a smaller distance relative to the amount of rotation of the advancement wheel 152 but such advancement or retraction may be accomplished with a reduced amount of force to the advancement wheel 152.

In some embodiments, the instrument delivery device 100 may include a seal (not illustrated) within the compartment 120 that isolates the spool drum 155a and the instrument 153 from the external environment. In some embodiments, the seal 122 may or may not be employed since fluid entering the instrument channel 121 would be prevented from escaping the compartment 120 by the seal within the compartment 120.

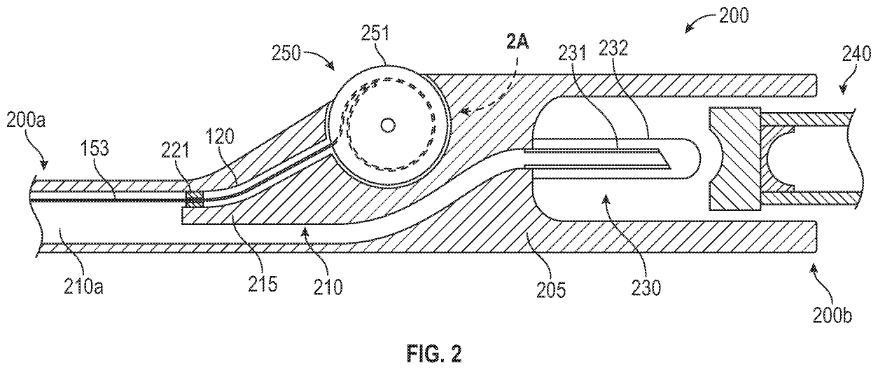

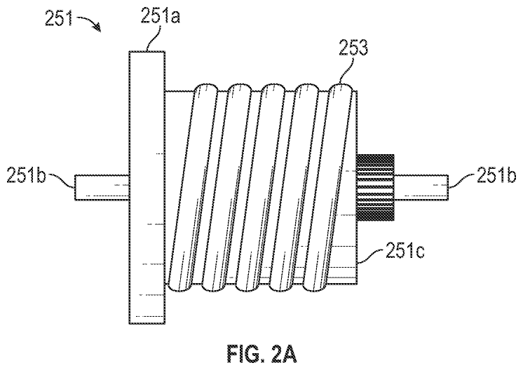

FIG. 2 illustrates another example of an instrument delivery device 200, in accordance with some embodiments. In some embodiments, the instrument delivery device 200 may be similar or identical to the instrument delivery device 100 in terms of one or more features and/or operation. In some embodiments, the instrument delivery device 200 may include a housing 205 having a distal end 200a, which may be configured in any manner described above, and a proximal end 200b that forms a vacuum tube receiver 230 having a needle 231 covered by a protective sheath 232. In some embodiments, a fluid pathway 210 may extend within the instrument delivery device 200 from the needle 231 to the distal end 200a. Accordingly, when a vacuum tube 240 is inserted into the vacuum tube receiver 230, a blood sample can be collected through the fluid pathway 210. In other embodiments, the proximal end 200b may include a Luer connector or any other type of connector that is coupled to the fluid pathway 210.

In some embodiments, the instrument delivery device 200 may include an instrument delivery mechanism 250 that enables an instrument 253 to be advanced in a distal direction through an IV catheter and/or subsequently withdrawn in a proximal direction. In some embodiments, a compartment 220 may be formed within the instrument delivery device 200 and may houses the instrument delivery mechanism 250. In some embodiments, a dividing wall 215 may create an instrument channel 221 that extends distally from the compartment 220 and joins the fluid pathway 210 at a distal portion 210a of the fluid pathway 210. In some embodiments, a seal 222 may be positioned within and the span the instrument channel 221 to isolate the instrument channel 221 from the fluid pathway 210.

As illustrated in FIG. 2A, which is an isolated rear view of the instrument delivery mechanism 250, the instrument delivery mechanism 250 may include a spool 251 having an axle 251b that maintains spool 251 within compartment 220 and allows spool 251 to rotate. In some embodiments, the spool 251 may include a spool drum 251c around which the instrument 253 is wound. In some embodiments, a portion of the spool 251 may form an advancement wheel 251a that extends upwardly from the compartment 220. Accordingly, a clinician can directly rotate the spool 251 by applying a force to the advancement wheel 251a. Such rotation can cause the instrument 253 to be advanced and retracted within the instrument channel 221 depending on the direction of rotation.

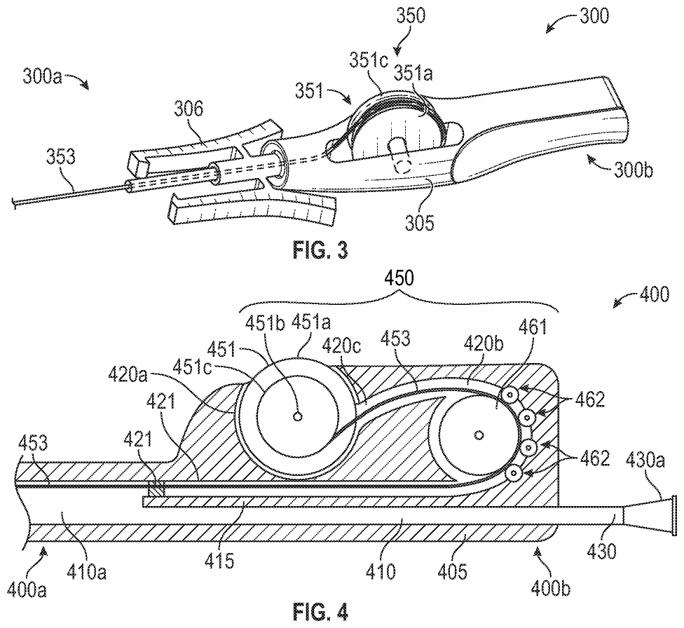

FIG. 3 illustrates another example of an instrument delivery device 300, in accordance with some embodiments. In some embodiments, the instrument delivery device 300 may be similar or identical to the instrument delivery device 100 and/or the instrument delivery device 200 in terms of one or more features and/or operation. In some embodiments, the instrument delivery device 300 may include a housing 305 having a distal end 300a and a proximal end 300b. In some embodiments, the distal end 300a may form a connector 306 by which the instrument delivery device 300 may be coupled to an IV catheter device (not illustrated). In some embodiments, the instrument delivery device 300 is an example of an instrument delivery device that is not configured to collect blood or inject fluid. Accordingly, in some embodiments, the proximal end 300b does not form a vacuum tube receiver or include an access port or other connector. In some embodiments, an instrument channel, but not a fluid pathway, may be formed within the housing 305. In some embodiments, the instrument 353 may be extended through the instrument channel, out through the distal end 300a, into an IV catheter device to which the instrument delivery device 300 is connected and ultimately through the IV catheter. It is noted, however, that in some embodiments, the proximal end 300b may be configured to allow blood to be collected or fluid to be injected using the instrument delivery device 300 (e.g., using any of the techniques described in the present disclosure).

In some embodiments, the instrument delivery device 300 may include an instrument delivery mechanism 350 having a spool 351 that is generally similar to the spool 251. In particular, the spool 351 may include an axle 351b that maintains the spool 351 within the compartment 320 and allows the spool 351 to rotate. In some embodiments, the spool 351 may include a spool drum 351c around which the instrument 353 is wound. In some embodiments, a portion of the spool 351 may form an advancement wheel 351a that extends upwardly from the compartment 320. Accordingly, a clinician can directly rotate the spool 351 by applying a force to the advancement wheel 351a and such rotation can cause the instrument 353 to advance or retract.

FIG. 4 illustrates another example of an instrument delivery device 400, in accordance with some embodiments. In some embodiments, the instrument delivery device 400 may be similar or identical to one or more of the following in terms of one or more features and/or operation: the instrument delivery device 100, the instrument delivery device 200, and the instrument delivery device 300. In some embodiments, the instrument delivery device 400 may include a housing 405 having a distal end 400a, which may be configured in any manner described above, and a proximal end 400b from which tubing 430 having a connector 430a extends. In some embodiments, a fluid pathway 410 may extend within the instrument delivery device 400 from the tubing 430 to a distal end 400a. Accordingly, a separate device could be coupled to the connector 430a to withdraw blood from or inject fluid into the fluid pathway 410. In some embodiments, the proximal end 400b may form a vacuum tube receiver similar to those described above. In some embodiments, the tube 430 may form a portion of the fluid pathway 410 (e.g., by extending distally up to the distal end of dividing wall 415).

In some embodiments, the instrument delivery device 400 may include an instrument delivery mechanism 450 that enables an instrument 453 to be advanced in a distal direction through an IV catheter and subsequently withdrawn in a proximal direction. In some embodiments, a distal compartment 420a and a proximal compartment 420b are formed within the instrument delivery device 400 and house the instrument delivery mechanism 450. In some embodiments, a compartment channel 420c interconnects the distal compartment 420a and the proximal compartment 420b. In some embodiments, a dividing wall 415 may create an instrument channel 421 that extends distally from the proximal compartment 420b and joins the fluid pathway 410 at a distal portion 410a of the fluid pathway 410. In some embodiments, a seal 422 may be positioned within and span the instrument channel 421 to isolate the instrument channel 421 from the fluid pathway 410.

In some embodiments, the instrument delivery mechanism 450 may include a spool 451 having an axle 451b that maintains the spool 451 within distal compartment 420a and allows the spool 451 to rotate. In some embodiments, the spool 451 may include a spool drum 451c around which the instrument 453 is wound. In some embodiments, a portion of the spool 451 may form an advancement wheel 451a that extends upwardly from the distal compartment 420a. Accordingly, a clinician can directly rotate spool 451 by applying a force to advancement wheel 451a.

In some embodiments, the instrument delivery mechanism 450 may include a primary wheel 461 having an axle 461a that maintains the primary wheel 461 within the proximal compartment 420b and allows the primary wheel 461 to rotate. In some embodiments, the instrument delivery mechanism 450 may further include one or more secondary wheels 462 that are adjacent to the primary wheel 461 and configured to rotate within proximal compartment 420b. In the depicted embodiment, there are four secondary wheels 462, but, in other embodiments, there may be a single secondary wheel or any other reasonable number of secondary wheels. Also, in some embodiments, instrument delivery mechanism 450 may include primary wheel 461 and no secondary wheels.

In some embodiments, the instrument 453 may be wound around the spool drum 451c and then extends proximally through the compartment channel 420c to wrap around the proximal side of the primary wheel 461. In some embodiments, each of the secondary wheels 462 can be positioned relative to the primary wheel 461 to retain the instrument 453 in close proximity, or even in constant contact with the primary wheel 461. Accordingly, the arrangement of the primary wheel 461 and the secondary wheels 462 may facilitate the advancement of the instrument 453 by reducing any resistance that may otherwise be caused as instrument wraps around the primary wheel 461. In some embodiments, more particularly, the secondary wheels 462 can hold the instrument 453 in contact with the primary wheel 461 so that, as the spool 451 is rotated to advance or retract the instrument 453, the advancement or retraction may cause the primary wheel 461 to rotate in unison. In some embodiments, as illustrated in FIG. 4, the instrument delivery mechanism 450 functions in a similar manner as a pulley system and can therefore reduce an amount of force that a clinician may need to apply to the spool 451 in order to advance or retract the instrument 453.

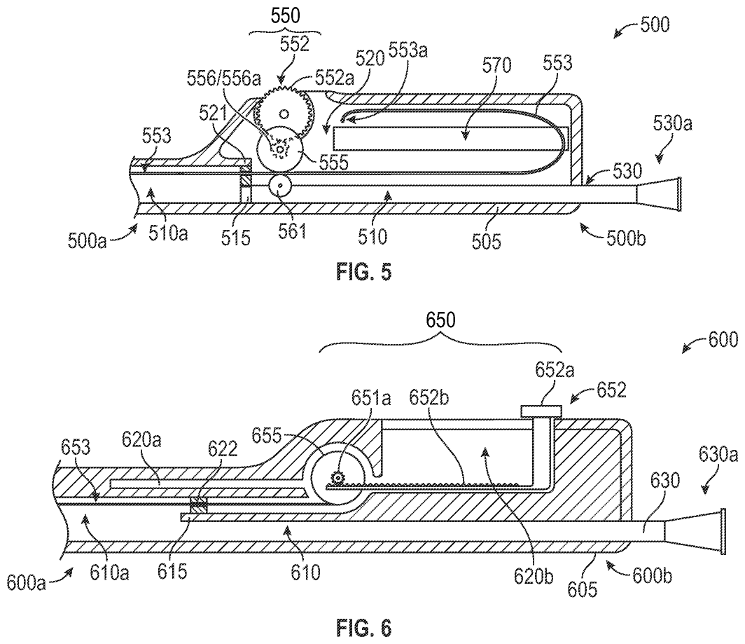

FIG. 5 illustrates another example of an instrument delivery device 500, in accordance with some embodiments. In some embodiments, the instrument delivery device 500 may be similar or identical to one or more of the following in terms of one or more features and/or operation: the instrument delivery device 100, the instrument delivery device 200, the instrument delivery device 300, and the instrument delivery device 400. In some embodiments, the instrument delivery device 500 may include a housing 505 having a distal end 500a, which may be configured in any manner described above, and a proximal end 500b from which tubing 530 having a connector 530a extends. In some embodiments, the tubing 530 may form the proximal portion of a fluid pathway 510 that extends within the instrument delivery device 500 up to the distal portion 510a of the fluid pathway 510. In some embodiments, because the tubing 530 forms the proximal portion of the fluid pathway 510, there may be no need for a dividing wall to separate the proximal portion of the fluid pathway 510 from compartment 520. However, in other embodiments, a dividing wall could be formed within the housing 505 similar to the embodiments described above. In other embodiments, the connector 530a could be replaced with a vacuum tube receiver similar to embodiments described above. In some embodiments, a seal 522 may be positioned within the housing 505 to isolate the compartment 520 from a distal portion 510a of the fluid pathway 510.

In some embodiments, the instrument delivery device 500 may include an instrument delivery mechanism 550 that enables an instrument 553 to be advanced in a distal direction through an IV catheter and subsequently withdrawn in a proximal direction. In some embodiments, the compartment 520 may be formed as a hollow interior of the housing 505. In some embodiments, the instrument delivery mechanism 550 may include a first guide wheel 555 having a gear 556 with teeth 556a, an advancement wheel 552 having teeth 552a along its outermost edge such that advancement wheel 552 functions as a gear that drives gear 556. In some embodiments, the instrument delivery mechanism 550 may further include a second guide wheel 561 that may be positioned below but adjacent to first guide wheel 555. In some embodiments, the instrument 553 may include an end 553a that is fixed to a portion of the housing 505 (e.g., a portion adjacent to advancement wheel 552). In some embodiments, the instrument 353 may be straight or curved or looped or configured in any way to facilitate easy advancement. In some embodiments, the instrument 553 may be initially routed in a proximal direction within the compartment 520 and then in a distal direction to pass between the first guide wheel 555 and the second guide wheel 561 and through the seal 522.

In some embodiments, the first guide wheel 555 and the second guide wheel 561 can be positioned close together so that the instrument 553 remains in contact with both guide wheels as instrument 553 is advanced or retracted. For example, the second guide wheel 561 could be biased against the first guide wheel 555. Accordingly, when a clinician rotates the advancement wheel 552, the gear formed by the advancement wheel 552 may cause the first guide wheel 555 to rotate. In some embodiments, because the instrument 553 is sandwiched between the first guide wheel 555 and the second guide wheel 561, the rotation of the first guide wheel 555 may cause the instrument 553 to be advanced or retracted depending on the direction of rotation. In some embodiments, the second guide wheel 561 can be configured to rotate to reduce any resistance caused as instrument 553 is advanced or retracted. In some embodiments, one or both of the first guide wheel 555 and the second guide wheel 561 can be formed of or coated with a high friction material so that the instrument 553 will not slide relative to the wheels (i.e., the friction will ensure that the wheels rotate as the instrument is advanced or retracted).

In some embodiments, including the depicted embodiment, the housing 505 may include a window 570 (e.g., a section of the housing that is transparent) that enables a clinician to see within the compartment 520. In some embodiments, the window 570 may enable the clinician to monitor the distance to which the instrument 553 has been advanced. For example, a clinician may look through the window 570 to see where the curved portion of the instrument 553 is positioned. In some embodiments, when this curved portion is positioned towards the proximal end 500b, the clinician can determine that the instrument 553 is fully retracted. In contrast, when the curved portion is positioned towards the distal end of the compartment 520, the clinician can determine that the instrument 553 is fully advanced. In some embodiments, the window 570 or another portion of the compartment 520 may include a ruler or other markings that represent the distance to which the instrument 553 has been advanced when the curved portion of the instrument 553 aligns with a particular marking. In some embodiments, the instrument 553 may be colored to increase its visibility within the window 570. In some embodiments, coloring of the instrument 553 may vary along its length such that the coloring can represent the distance to which the instrument 553 has been advanced.

FIG. 6 illustrates another example of an instrument delivery device 600, in accordance with some embodiments. In some embodiments, the instrument delivery device 600 may be similar or identical to one or more of the following in terms of one or more features and/or operation: the instrument delivery device 100, the instrument delivery device 200, the instrument delivery device 300, the instrument delivery device 400, and the instrument delivery device 500. In some embodiments, the instrument delivery device 600 may include a housing 605 having a distal end 600a, which may be configured in any manner described above, and a proximal end 600b from which tubing 630 having a connector 630a extends. In some embodiments, a fluid pathway 610 may extend within the instrument delivery device 600 from tubing 630 to the distal end 600a.

In some embodiments, the instrument delivery device 600 may include an instrument delivery mechanism 650 that enables an instrument 653 to be advanced in a distal direction through an IV catheter and subsequently withdrawn in a proximal direction. In some embodiments, a compartment 620/620a/620b is formed within instrument delivery device 600 and houses the instrument delivery mechanism 650. In some embodiments, a dividing wall 615 may create an instrument channel 621 that extends distally from compartment 620 and joins fluid pathway 610 at a distal portion 610a of fluid pathway 610. In some embodiments, a seal 622 may be positioned within and span the instrument channel 621 to isolate the instrument channel 621 from the fluid pathway 610.

In some embodiments, the instrument delivery mechanism 650 may include a pinion 655 that is configured to rotate within the compartment 620. In some embodiments, the instrument delivery mechanism 650 may also include a rack mechanism 652 having a rack 652b and an actuator portion 652a. In some embodiments, the actuator portion 652a may extend out from proximal portion 620b of the compartment 620 to thereby enable a clinician to use his or her thumb or finger to slide the rack mechanism 652 along the proximal portion 620b and distal portion 620a of compartment 620. In some embodiments, the rack 652b may be positioned to interface with the pinion 655 so that the pinion 655 rotates as the rack 652b is slid laterally. In some embodiments, the pinion 655 may include a spool drum (not visible) around which the instrument 653 may be wound. This spool drum of the pinion 655 can be similar to those described above. Accordingly, as the rack mechanism 652 is slid distally, the instrument 653 may be advanced distally. Likewise, as the rack mechanism 652 is slid proximally, the instrument 653 may be withdrawn proximally. In some embodiments, the position of actuator portion 652a can therefore represent the distance to which the instrument 653 is advanced. In some embodiments, a ruler or other markings may be formed on a portion of housing 605 along which actuator portion 652a slides.

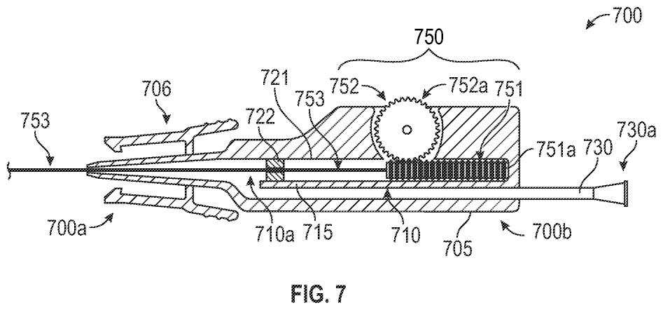

FIG. 7 illustrates another example of an instrument delivery device 700, in accordance with some embodiments. In some embodiments, the instrument delivery device 700 may be similar or identical to one or more of the following in terms of one or more features and/or operation: the instrument delivery device 100, the instrument delivery device 200, the instrument delivery device 300, the instrument delivery device 400, the instrument delivery device 500, and the instrument delivery device 600. In some embodiments, the instrument delivery device 700 may include a housing 705 having a distal end 700a forming a connector 706 and a proximal end 700b from which tubing 730 having a connector 730a extends. In some embodiments, a fluid pathway 710 may extend within the instrument delivery device 700 from tubing 730 to distal end 700a.

In some embodiments, the instrument delivery device 700 may include an instrument delivery mechanism 750 that enables an instrument 753 to be advanced in a distal direction through an IV catheter and subsequently withdrawn in a proximal direction. In some embodiments, a compartment 720 is formed within the instrument delivery device 700 and houses the instrument delivery mechanism 750. In some embodiments, a dividing wall 715 creates an instrument channel 721 that extends distally from the compartment 720 and joins the fluid pathway 710 at a distal portion 710a of the fluid pathway 710. In some embodiments, a seal 722 may be positioned within and span the instrument channel 721 to isolate the instrument channel 721 from the fluid pathway 710.

In some embodiments, the instrument delivery mechanism 750 may include a shaft 751 that is positioned at a proximal end of instrument channel 721 and an advancement wheel 752 that is positioned within compartment 720 and extends out from housing 705. In some embodiments, the advancement wheel 752 may include teeth 752a that interface with teeth 751a of the shaft 751. Accordingly, when a clinician rotates the advancement wheel 752, the shaft 751 will be moved linearly within the instrument channel 721. In some embodiments, the instrument 753 can be secured within the shaft 751 so that, when the shaft 751 is moved linearly, the instrument 753 will advance distally or withdraw proximally depending on the direction of rotation of the advancement wheel 752.

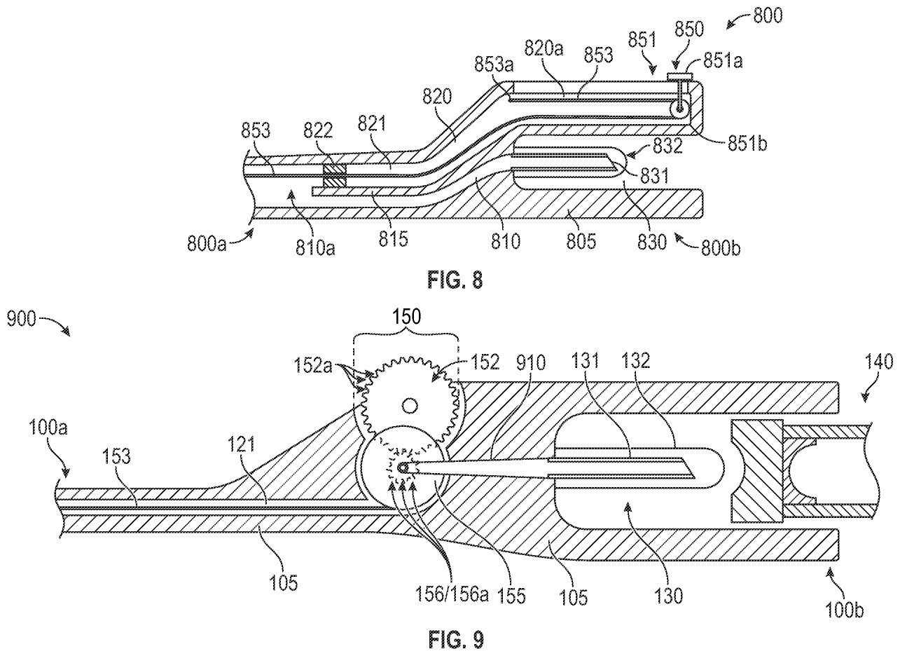

FIG. 8 illustrates another example of an instrument delivery device 800, in accordance with some embodiments. In some embodiments, the instrument delivery device 800 may be similar or identical to one or more of the following in terms of one or more features and/or operation: the instrument delivery device 100, the instrument delivery device 200, the instrument delivery device 300, the instrument delivery device 400, the instrument delivery device 500, the instrument delivery device 600, and the instrument delivery device 700. In some embodiments, the instrument delivery device 800 may include a housing 805 having a distal end 800a, which may be configured in any manner described above, and a proximal end 800b that forms a vacuum tube receiver 830 having a needle 831 covered by a protective sheath 832. In some embodiments, a fluid pathway 810 may extend within the instrument delivery device 800 from needle 831 to distal end 800a.

In some embodiments, the instrument delivery device 800 may include an instrument delivery mechanism 850 that enables an instrument 853 to be advanced in a distal direction through an IV catheter and subsequently withdrawn in a proximal direction. In some embodiments, a compartment 820 is formed within the instrument delivery device 800 and houses the instrument delivery mechanism 850. In some embodiments, a dividing wall 815 may create an instrument channel 821 that extends distally from compartment 820 and joins the fluid pathway 810 at a distal portion 810a of the fluid pathway 810. In some embodiments, a seal 822 may be positioned within and span the instrument channel 821 to isolate the instrument channel 821 from the fluid pathway 810.

In some embodiments, the instrument delivery mechanism 850 may include a sliding member 851 having an actuator portion 851a that extends out from the compartment 820 and a wheel 851b that is connected to the actuator portion 851a. In some embodiments, the compartment 820 can be configured to allow the sliding member 851 to slide distally and proximally within the compartment 820 when a clinician applies a force to the actuator portion 851a. In some embodiments, an end 853a of the instrument 853 can be fixed to the housing 805 towards a distal end of compartment 820. In some embodiments, the instrument 853 is initially routed in a proximal direction around wheel 851b and then in a distal direction through instrument channel 821. In some embodiments, the wheel 851b can be configured to retain the instrument 853 in contact with the wheel 851b even while the sliding member 851 is slid within the compartment 820 (e.g., using retaining bars (not illustrated) that perform a similar function as the secondary wheels 462). Accordingly, as the sliding member 851 is slid distally, wheel 851b can rotate to thereby advance the instrument 853 in a distal direction. In some embodiments, because the instrument 853 is “folded back” on itself, the instrument 853 will be advanced/retracted twice the distance that sliding member 851 is advanced/retracted.

A number of variations have been described in the context of particular depicted embodiments. It is noted that, even though not explicitly described for each depicted embodiment, such variations may apply to any of the above-described or depicted embodiments. A number of additional variations may also be made as is now described.

In some embodiments, an instrument delivery mechanism may include a spring or other mechanism that becomes loaded as the instrument is advanced. In such embodiments, the instrument delivery mechanism may also include a ratcheting or locking mechanism that prevents the spring from unloading until the clinician releases the ratcheting or locking mechanism (e.g., by pressing a button). Once released, the spring can cause the instrument to automatically retract. For example, in the context of FIG. 2, a spring and ratcheting mechanism may be incorporated into the spool 251, and a release button may be integrated into the housing 205. In such cases, as the spool 251 is rotated to advance the instrument 253, the spring will become loaded. Once the clinician desires to withdraw the instrument 253, he or she may press the button to release the ratcheting mechanism. The loaded spring will then cause the spool 251 to rotate in the reverse direction to cause the instrument 253 to be wound back around the spool drum 251c. Similar techniques could be employed on any of the embodiments that employ a wheel, spool or other rotating member. With regards to the instrument delivery mechanism 650 and the instrument delivery mechanism 850, a linear spring could be loaded as the respective actuator portion is slid distally and unloaded in response to the clinician releasing a ratcheting or other locking mechanism.

In any of described embodiments, the instrument delivery device may include some type of indicia representing how far the instrument is advanced. Such indicia could be passive (e.g., ruler markings, labels, colors, scales, numbers, symbols, etc.) or active (e.g., a digital display, a speaker, etc.). Also, in any of the described embodiments, the instrument delivery mechanism may include mechanisms for preventing over-advancement or over-retraction of the instrument. For example, the embodiments that employ rotating components could include stops that contact the rotating components when the instrument has reached its maximum advancement distance. As suggested above, although the depicted embodiments show the fluid pathway extending to the proximal end of the instrument delivery device, in some embodiments, the fluid pathway could extend out from the instrument delivery device at a point other than the proximal end including towards a distal end of the instrument delivery device. As an example only, the fluid pathway 110 could extend out of the instrument delivery device 100 towards the distal portion 110a at a point opposite the instrument delivery mechanism 150 to form a vacuum tube receiver or other connector.

In any of the described embodiments, the fluid pathway and the instrument channel may be the same pathway/channel. For example, FIG. 9 illustrates an instrument delivery device 900 that resembles the instrument delivery device 100 except that the instrument delivery device 900 does not include the fluid pathway 110 or the seal 122. Instead, the instrument channel 121 forms the distal portion of the fluid pathway. In some embodiments, a proximal portion 910 of the fluid pathway can extend from the spool 155 to the needle 131 (or any other connector/adapter that may be employed). In such embodiments, the instrument 153 may be in the form of a tube such that the instrument 153 forms the proximal portion 910 of the fluid pathway. In other words, the proximal end of the instrument 153 may extend proximally (or in some other direction) from the spool 155 to connect to the needle 131. In other variations, separate tubing may extend from the instrument channel or the compartment that houses the instrument delivery mechanism to form the proximal end of the fluid pathway. For example, a separate tube or channel could be formed from compartment 520, 620 or 820 to form a fluid pathway to a vacuum tube receiver or other connector.

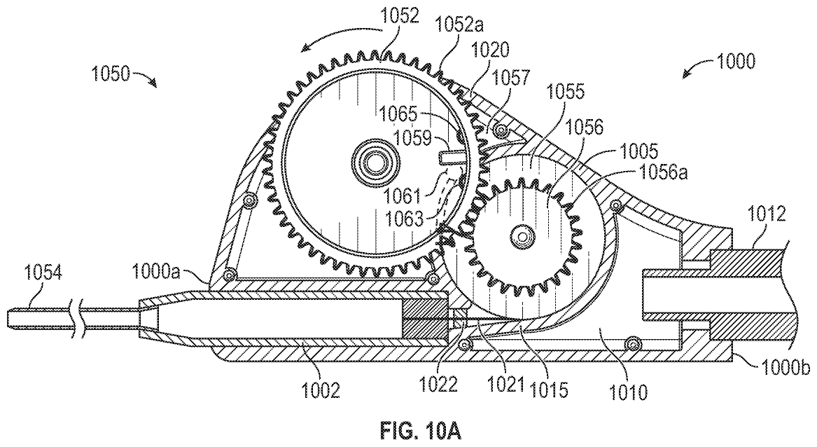

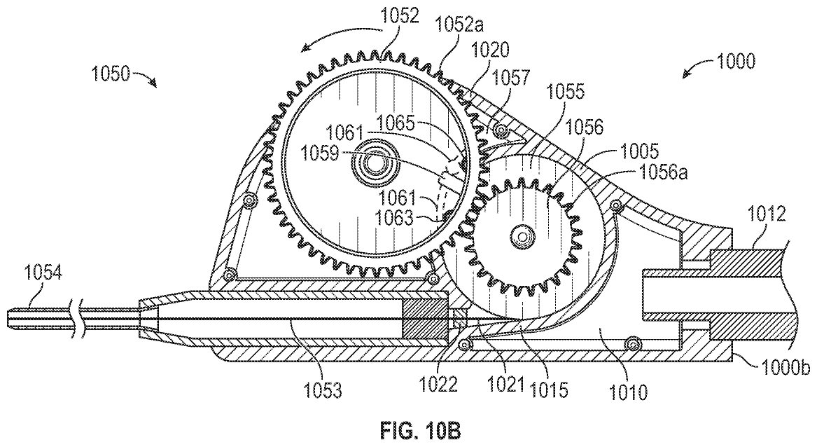

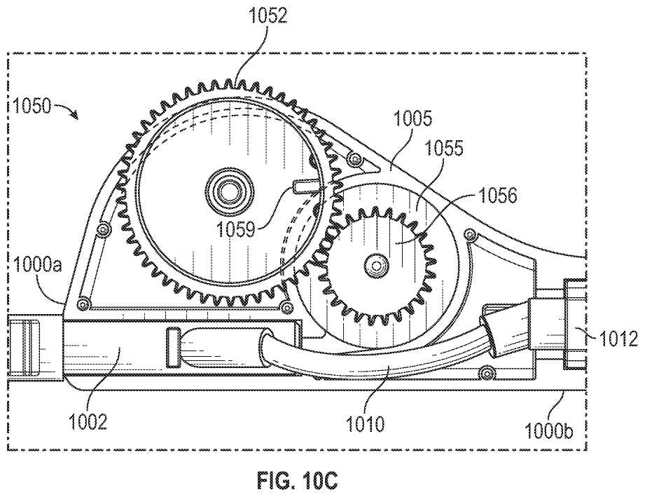

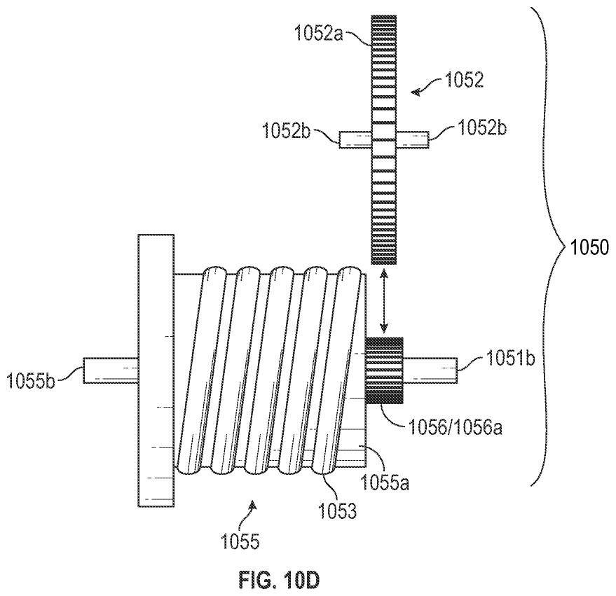

FIGS. 10A-10C illustrate another example of an instrument delivery device 1000, in accordance with some embodiments. In some embodiments, the instrument delivery device 1000 may be similar or identical to one or more of the following in terms of one or more features and/or operation: the instrument delivery device 100, the instrument delivery device 200, the instrument delivery device 300, the instrument delivery device 400, the instrument delivery device 500, the instrument delivery device 600, the instrument delivery device 700, the instrument delivery device 800, and the instrument delivery device 900.

In some embodiments, the instrument delivery device 1000 may include a housing 1005, which may include a distal end 1000a and a proximal end 1000b. In some embodiments, the distal end 1000a may include any type of connector to enable the instrument delivery device 1000 to be connected to an IV catheter device 1002 or could incorporate an IV catheter. In some embodiments, the proximal end 1000b may be configured to form a vacuum tube receiver, which may include a needle covered by a protective sheath (see, for example, FIG. 1). In some embodiments, as illustrated in FIG. 10B, the proximal end 1000b may include a Luer connector or another suitable type of connector that couples to a blood collection device 1012.

In some embodiments, a fluid pathway 1010 may extend within the instrument delivery device 1000 through the distal end 1000a and the proximal end 1000b of the housing 1005. Accordingly, when the blood collection device 1012 is coupled to the proximal end 1000b, a blood sample can be collected through the fluid pathway 1010. In some embodiments, the fluid pathway 1010 may be configured to connect a side port of the IV catheter device 1002 with the blood collection device 1012 and may be disposed laterally to the cross-section of FIGS. 10A-10B. In some embodiments, the fluid pathway 1010 may extend through a tube coupled to and/or integrated with the side port of the IV catheter device 1002 and the blood collection device 1012, as illustrated, for example, in FIG. 10C. In FIG. 10C, the housing 1005 is transparent, according to some embodiments, which allows illustration of internal components of the housing 1005.

In some embodiments, the instrument delivery device 1000 may include an instrument delivery mechanism 1050 that enables an instrument 1053 to be advanced in a distal direction through an IV catheter 1054 and/or subsequently withdrawn in a proximal direction. In some embodiments, the instrument 1053 may include a wire constructed of nickel titanium or another suitable material. In some embodiments, a compartment 1020 may be formed within the instrument delivery device 1000 and may house the instrument delivery mechanism 1050. In some embodiments, a dividing wall 1015 may create an instrument channel 1021 that extends distally from the compartment 1020 and joins the fluid pathway 1010 at a distal portion 1010a of the fluid pathway 1010.

In some embodiments, to isolate the compartment 1020 from the fluid pathway 1010, a seal 1022 (e.g., an elastomeric septum) may be positioned within and span the instrument channel 1021. In some embodiments, the instrument 1053 may extend through a slit or other opening formed within seal 1022. In some embodiments, the seal 1022 may provide support to instrument 1053 to prevent it from buckling as it is advanced.

In some embodiments, the instrument delivery mechanism 1050 may include a spool 1055 and an advancement wheel 1052, both of which may be configured to rotate within the compartment 1020. In some embodiments, the spool 1055 may be positioned adjacent to the advancement wheel 1052 (e.g., towards the instrument channel 1021 relative to the advancement wheel 1052). In some embodiments, the advancement wheel 1052 may be positioned to extend partially out from the compartment 1020 to thereby enable a clinician to use his or her thumb or finger to rotate the advancement wheel 1052. In some embodiments, the spool 1055 may include a gear 1056 having teeth 1056a. Likewise, in some embodiments, the advancement wheel 1052 may include teeth 1052a and may therefore function as a gear. In some embodiments, the teeth 1052a may interface with the teeth 1056a so that the spool 1055 is rotated when the advancement wheel 152 is rotated. In some embodiments, the teeth 1052a may be formed along the outermost edge of the advancement wheel 1052. In other embodiments, however, teeth 1052a may be formed along a portion of advancement wheel that is inset relative to the outermost edge.

FIG. 10D provides an exploded rear view of the instrument delivery mechanism 1050 in isolation, according to some embodiments. In some embodiments, the spool 1055 and the advancement wheel 152 may include axles 1055b and 1052b, respectively, by which these components are positioned within the compartment 1020 and around which these components rotate. In some embodiments, the spool 1055 may include a spool drum 1055a around which the instrument 1053 may be wound. Therefore, when the spool 1055 is rotated, the rotation may cause the instrument 1053 to be advanced or retracted along the instrument channel 1021 depending on the direction in which the advancement wheel 1052 is rotated. In some embodiments, the gear formed by the advancement wheel 1052 may have a larger diameter than the gear 1056 to thereby cause the instrument 1053 to be advanced or retracted a larger distance relative to the amount of rotation of advancement wheel 1052. In contrast, in other embodiments, the gear formed by the advancement wheel 1052 may have an equal or smaller diameter than the gear 1056. In such embodiments, the instrument 1053 may advance or retract a smaller distance relative to the amount of rotation of the advancement wheel 1052 but such advancement or retraction may be accomplished with a reduced amount of force to the advancement wheel 1052.

In some embodiments, the instrument delivery device 1000 may include a seal (not illustrated) within the compartment 1020 that isolates the spool drum 1055a and the instrument 1053 from the external environment. In some embodiments, the seal 1022 may or may not be employed since fluid entering the instrument channel 1021 may be prevented from escaping the compartment 1020 by the seal within the compartment 1020.

In some embodiments, the instrument delivery device 1000 may facilitate needle-free delivery of an instrument 1219 into vasculature of the patient for blood collection, fluid delivery, patient or device monitoring, or other clinical needs by utilizing an existing vascular access device dwelling within the vasculature of the patient, such as the IV catheter device 1002. In some embodiments, the instrument delivery device 1000 may reduce trauma to the vein, decrease fill time, and overcome thrombus and fibrin sheath in or around the vascular access device that may otherwise prevent blood draw.

In some embodiments, the spool 1055 may be turned or allowed to turn to advance the instrument 1053 in a distal direction. In some embodiments, it is important that there is a means to stop the spool 1055 and/or the advancement wheel 1052. In further detail, in some embodiments, the advancement wheel 1052 may include a stop member 1059 and the housing 1005 may include another stop member 1061. In some embodiments, the stop member 1059 may be disposed on an outer surface of the advancement wheel 1052 and/or the other stop member 1061 may be disposed on an inner surface 1057 of the housing 1005. In some embodiments, the stop member 1059 and/or the other stop member 1061 may include a protrusion or another element configured to contact or interfere with each other and stop further rotation of the advancement wheel 1052. In some embodiments, the stop member 1059 and the other stop member 1061 may be configured to contact each other to stop rotation of the advancement wheel 1052 and the spool 1055.

FIG. 10A illustrates the advancement wheel 1052 and the instrument 1053 in a first configuration, according to some embodiments. FIG. 10B illustrates the advancement wheel 1052 and the instrument 1053 in a second configuration, in which the instrument 1053 is advanced, according to some embodiments. In some embodiments, the advancement wheel 1052 may be rotated between the first configuration and the second configuration but may be prevented from completing a full turn by contact between the stop member 1059 and the other stop member 1061, which may stop rotation of the advancement wheel 1052. In some embodiments, the advancement wheel 1052 may be rotated in an opposite direction from the second configuration to the first configuration to retract the instrument 1053 after use. In some embodiments, the advancement wheel 1052 may be prevented from rotating more than 360 degrees because of contact between the stop member 1059 and the other stop member 1061.

In some embodiments, the advancement wheel may be rotated in a first direction from the first configuration to the second configuration. In some embodiments, in the first configuration, the stop member 1059 and the other stop member 1061 may contact each other to stop rotation of the advancement wheel 1052 in a second direction opposite the first direction. In some embodiments, in the second configuration, the stop member 1059 and the other stop member 1061 may be configured to contact each other to prevent further rotation of the advancement wheel 1052 in the first direction, thereby stopping advancement of the instrument 1053 in the distal direction.

In some embodiments, the outer surface of the advancement wheel 1052 may include a bump 1063 and/or another bump 1065 that may be spaced apart from the stop member 1059. In some embodiments, a width of the other stop member 1061 may be approximately equal to or slightly less than a space between the bump 1063 and the stop member 1059 and/or a space between the other bump 1065 and the stop member 1059. Thus, in some embodiments, the other stop member 1061 may fit snugly between the bump 1063 and the stop member 1059 and/or between the other bump 1065 and the stop member 1059. In some embodiments, the bump 1063 and/or the other bump 1065 may provide some securement of the instrument 1053 in the advanced position and/or the retracted position.

In some embodiments, the bump 1063 may provide resistance to movement and turning of the advancement wheel 1052 when the advancement wheel 1052 is in the first configuration. In some embodiments, the other bump 1065 may provide resistance to movement and turning of the advancement wheel 1052 when the advancement wheel 1052 is in the second configuration. In some embodiments, the bump 1063 and/or the other bump 1065 may each have a width and/or a height less than that of the stop member 1059 such that the resistance to movement and turning of the advancement wheel 1052 provided by the bump 1063 and/or the other bump 1065 can be overcome, for example, to turn the advancement wheel 1052 from the first configuration to the second configuration and from the second configuration to the first configuration. In some embodiments, the stop member 1059 may extend inwardly further than the other stop member 1061 to facilitate blocking passage of the other stop member 1061,

It is understood that in some embodiments, the bump 1063 and/or the other bump 1065 may be disposed on the inner surface of the housing 1005 to perform a same or similar function. In these embodiments, the bump 1063 and/or the other bump 1065 may be spaced apart from the other stop member 1061, and a width of the stop member 1059 may be approximately equal to or slightly less than a space between the bump 1063 and the other stop member 1061 and/or a space between the other bump 1065 and the other stop member 1061. It is also understood that the bump 1063, the other bump 1065, or an additional bump or bumps may be placed at alternate locations on the outer surface of the advancement wheel 1052 and/or the inner surface of the housing 1005. In these embodiments, one or more of the bump 1063, the other bump 1065, and the additional bump or bumps may signal to the clinician that the advancement wheel 1052 and therefore the spool 1055 and the instrument 1053 are in a particular position.

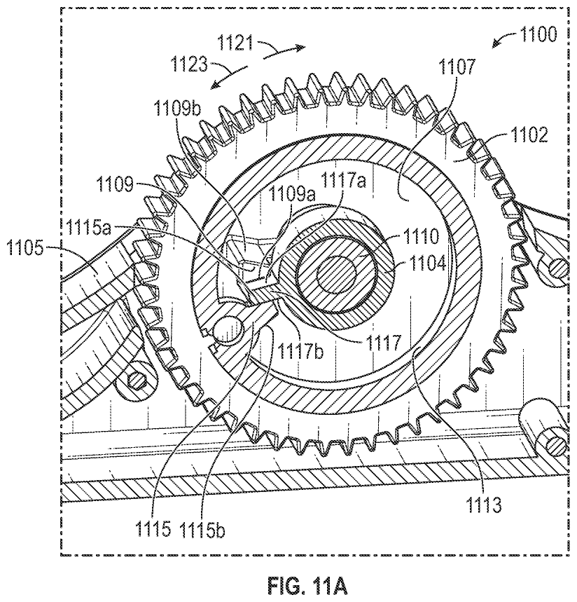

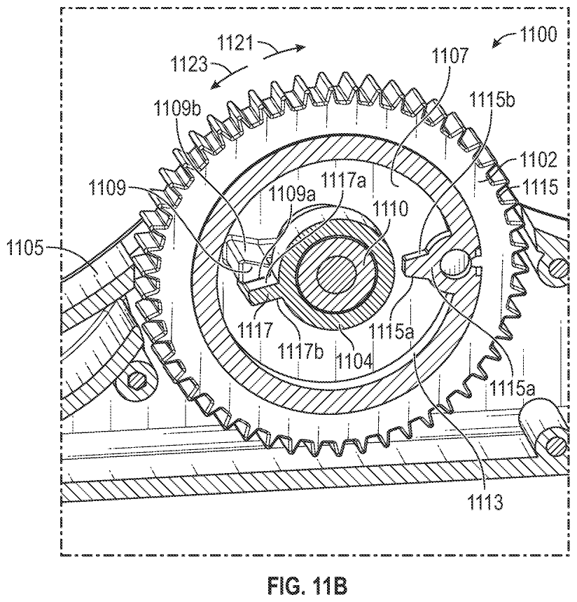

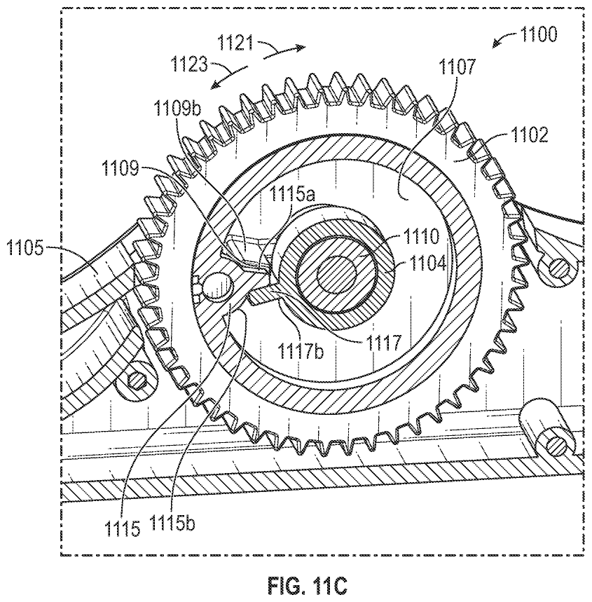

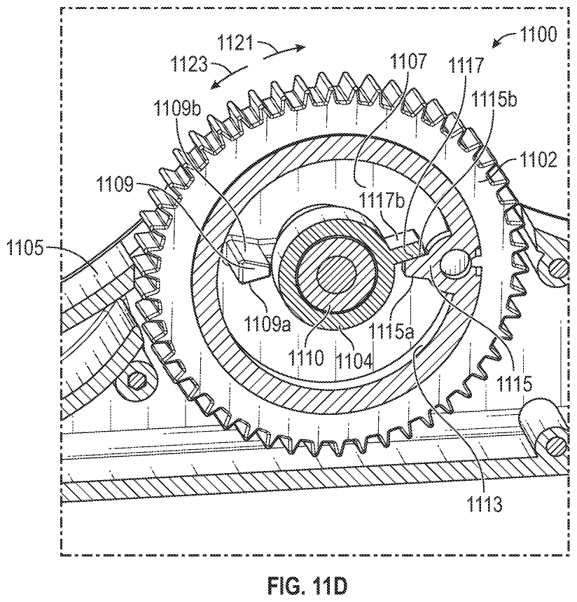

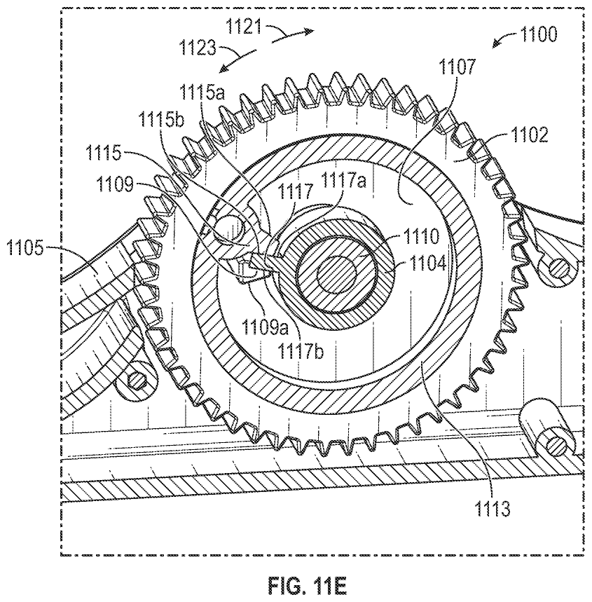

FIGS. 11A-11E illustrate another example of an instrument delivery device 1100, in accordance with some embodiments. In some embodiments, the instrument delivery device 1100 may be similar or identical to one or more of the following in terms of one or more features and/or operation: the instrument delivery device 100, the instrument delivery device 200, the instrument delivery device 300, the instrument delivery device 400, the instrument delivery device 500, the instrument delivery device 600, the instrument delivery device 700, the instrument delivery device 800, the instrument delivery device 900, and the instrument delivery device 1000. In some embodiments, the instrument delivery device 1100 may be moved from FIG. 11A to FIG. 11B to FIG. 11C to FIG. 11D to FIG. 11E.

In some embodiments, the instrument delivery device 1100 may include a first wheel 1102 and a second wheel 1104. In some embodiments, the first wheel 1102 of the instrument delivery device 1100 may include or correspond to the advancement wheel 152 of FIG. 1, the spool 155 of FIG. 1, the spool 251 of FIG. 2, the spool 351 of FIG. 3, the spool 451 of FIG. 4, the advancement wheel 552 of FIG. 5, the first guide wheel 555 of FIG. 5, the pinion 655 of FIG. 6, the advancement wheel 752 of FIG. 7, or the advancement wheel 1052 of FIG. 10.

In some embodiments, the instrument delivery device 1100 may include a housing 1105, which may include a distal end and a proximal end. In some embodiments, the distal end of the housing 1105 may be configured to couple to an IV catheter device. In some embodiments, the housing 1105 of the instrument delivery device 1100 may include or correspond to the housing 105 of FIG. 1, the housing 205 of FIG. 2, the housing 305 of FIG. 3, the housing 405 of FIG. 4, the housing 505 of FIG. 5, the housing 605 of FIG. 6, the housing 705 of FIG. 7, the housing 805 of FIG. 8, or the housing 1005 of FIG. 10.

In some embodiments, an inner surface 1107 of the housing 1105 may include a housing stop member 1109, which may include a protrusion. In some embodiments, the housing stop member 1109 may include a first side 1109a and a second side 1109b, which may be opposite the first side 1109a.

In some embodiments, the first wheel 1102 may rotate on an axle 1111. In some embodiments, the first wheel 1102 may include a protrusion or rod 1110, which may rotate on the axle 1111. In some embodiments, the axle 1111 may be aligned with a center axis of the first wheel 1102. In some embodiments, the second wheel 1104 may be disposed on the rod 1110 and configured to slip with respect to the rod 1110 and/or rotate independently from the rod 1110. In some embodiments, the second wheel 1104 may ride directly on the axle 1111, along with the first wheel 1102. In some embodiments, the axle 1111 may extend inwardly from the housing 1105. In some embodiments, the first wheel 1102 and the second wheel 1104 may rotate about a same axis and/or the second wheel 1104 may be disposed within the first wheel 1102.

In some embodiments, an inner surface 1113 of the first wheel 1102 may include a first wheel stop member 1115, which may include a protrusion. In some embodiments, a gap may be disposed between the housing stop member 1109 and the first wheel stop member 1115. In some embodiments, the second wheel 1104 may include a tab 1117, which may be configured to bridge the gap between the housing stop member 1109 and the first wheel stop member 1115.

In some embodiments, the instrument delivery device 1100 may include an instrument. In some embodiments, the housing instrument of the instrument delivery device 1100 may include or correspond to the instrument 153 of FIG. 1, the instrument 253 of FIG. 2, the instrument 353 of FIG. 3, the instrument 453 of FIG. 4, the instrument 553 of FIG. 5, the instrument 653 of FIG. 6, the instrument 753 of FIG. 7, the instrument 853 of FIG. 8, or the instrument 1053 of FIG. 10.

In some embodiments, the first wheel 1102 may be configured to rotate to advance the instrument in a distal direction through the distal end of the housing 1105. In some embodiments, the first wheel 1102 may be configured to rotate more than one full turn. In some embodiments, the instrument delivery device 1100 may be disposed in a first configuration, as illustrated, for example, in FIG. 11A.

In some embodiments, in response to the instrument delivery device 1100 being disposed in the first configuration, the tab 1117 may be disposed between the housing stop member 1109 and the first wheel stop member 1115 and may contact the housing stop member 1109 and the first wheel stop member 1115. In these embodiments, a first side 1117a of the tab 1117 may contact a first side 1109a of the housing stop member 1109 and a second side 1117b of the tab 1117 may contact a first side 1115a of the first wheel stop member 1115. In some embodiments, in response to the instrument delivery device 1100 being disposed in the first configuration, the first wheel 1102 and/or the second wheel 1104 may be prevented from rotating in a first direction 1121, toward the housing stop member 1109, but may be configured to rotate in a second direction 1123 opposite the first direction 1121. In some embodiments, the first wheel 1102 may be configured to rotate more than one full turn in the second direction.

In some embodiments, in response to the instrument delivery device 1100 being disposed in the first configuration, the first wheel 1102 may be configured to rotate independent from the housing 1105 and the second wheel 1104 in a second direction 1123 until the first wheel stop member 1115 contacts the tab 1117. In these embodiments, the first wheel 1102 may be configured to rotate independent from the housing 1105 and the second wheel 1104 in the second direction 1123 opposite the first direction 1121 until a second side 1115b of the first wheel stop member 1115 contacts the first side 1117a of the tab 1117, which may be disposed on an opposite side of the tab 1117 as the second side 1117b. In these embodiments, the first wheel 1102 may be configured to rotate almost one full turn independent from the housing 1105 and the second wheel 1104.

In some embodiments, the instrument may be in a retracted or fully retracted position in response to the instrument delivery device 1100 being in the first configuration. In some embodiments, in response to the first wheel 1102 rotating independent from the housing 1105 and the second wheel 1104 in the second direction 1123 until the first wheel stop member 1115 contacts the second side 1117b of the tab 1117, the first wheel 1102 and the second wheel 1104 are configured to rotate together further in the second direction 1123 until the instrument delivery device 1100 is disposed in a second configuration. In these embodiments, the first wheel 1102 and the second wheel 1104 may be configured to rotate together almost one full turn.

In some embodiments, the instrument may be in an advanced or fully advanced position in response to the instrument delivery device 1100 being in the second configuration. In some embodiments, in the second configuration, the tab 1117 may be disposed between the housing stop member 1109 and the first wheel stop member 1115 and may contact the housing stop member 1109 and the first wheel stop member 1115. In these embodiments, the second side 1117b of the tab 1117 may contact the second side 1109b of the housing stop member 1109 and the first side 1117a of the tab 1117 may contact the second side 1115b of the first wheel stop member 1115, as illustrated, for example, in FIG. 11E.

In some embodiments, FIG. 11B illustrates the first wheel 1102 rotating in the second direction 1123 independently from the first configuration. In some embodiments, the first wheel 1102 may rotate in the second direction 1123 independently from the first configuration until the first side 1115a of the first wheel stop member 1115 contacts the first side 1117a of the tab 1117, as illustrated in FIG. 11C, for example. In some embodiments, FIG. 11D illustrates the first wheel 1102 and the second wheel 1104 may rotate together further in the second direction 1123, as illustrated in FIG. 11D, for example. In some embodiments, the first wheel 1102 and the second wheel 1104 may rotate together until the second side 1117b of the tab 1117 contacts the second side 1109b of the housing stop member 1109, as illustrated, for example, in FIG. 11E, preventing further rotation in the second direction 1123.

In some embodiments, the first wheel 1102 may be configured to rotate almost two full turns, from the first configuration to the second configuration. In is understood that in some embodiments, the instrument delivery device 1100 one or more other wheels that operate in a similar fashion to the second wheel 1104 to each allow almost another full turn of the first wheel 1102. In these embodiments, the one or more other wheels may be disposed between the second wheel 1104 and the housing stop member 1109.

In some embodiments, the first wheel 1102 may extend out from the housing 1105, which may facilitate turning of the first wheel 1102 by a digit of the clinician. In some embodiments, in order to advance the instrument, the clinician may rotate the portion of the first wheel 1102 exposed from the housing 1105 toward the distal end 1100a of the housing 1105 or in the second direction 1123 to advance the instrument distally. In some embodiments, the clinician may rotate the portion of the first wheel 1102 exposed from the housing 1105 away from the distal end 1100a of the housing 1105 or in the first direction 1121 to retract the instrument proximally.

It is understood, however, in some embodiments, positions of the housing stop member 1109 and the first wheel stop member 1115 may be reversed. In these embodiments, the clinician may rotate the portion of the first wheel 1102 exposed from the housing 1105 away from the distal end 1100a of the housing 1105 or in the first direction 1121 to advance the instrument distally and/or the clinician may rotate the portion of the first wheel 1102 exposed from the housing 1105 away from the distal end 1100a of the housing 1105 or in the first direction 1121 to retract the instrument proximally. In some embodiments, a location of the housing stop member 1109, the tab 1117, and the first wheel stop member 1115 in the first configuration may vary.

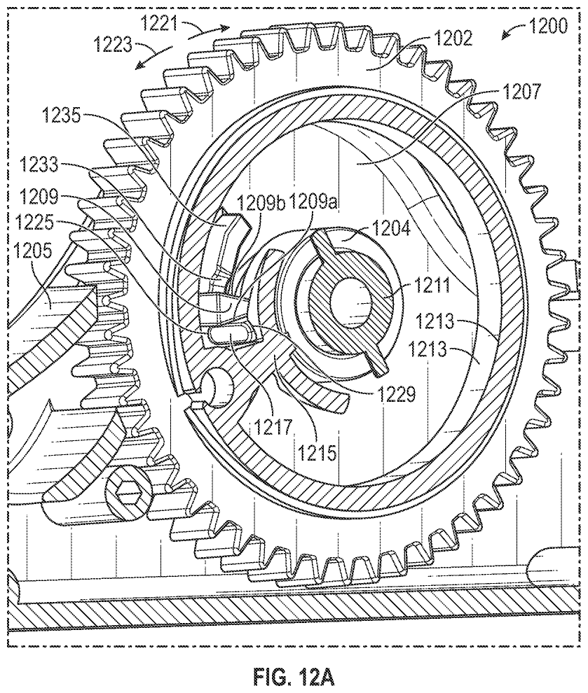

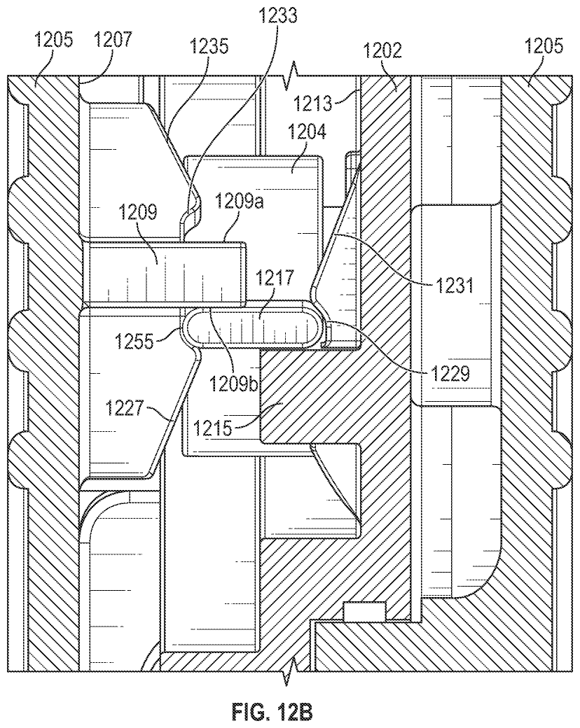

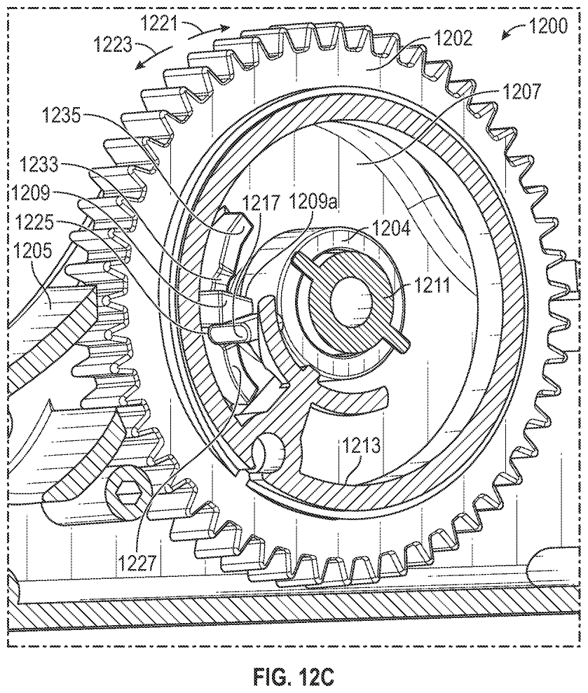

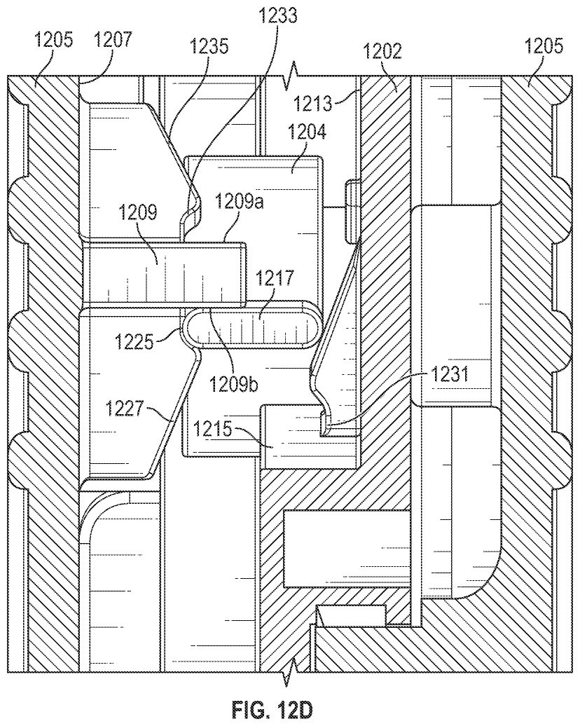

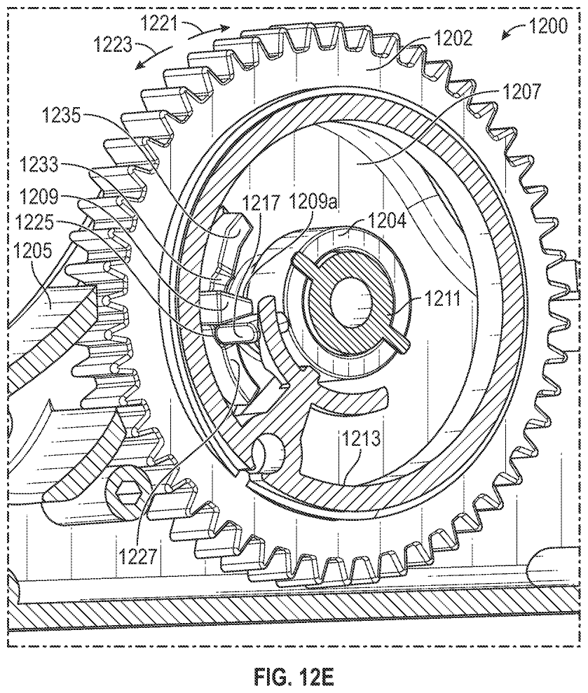

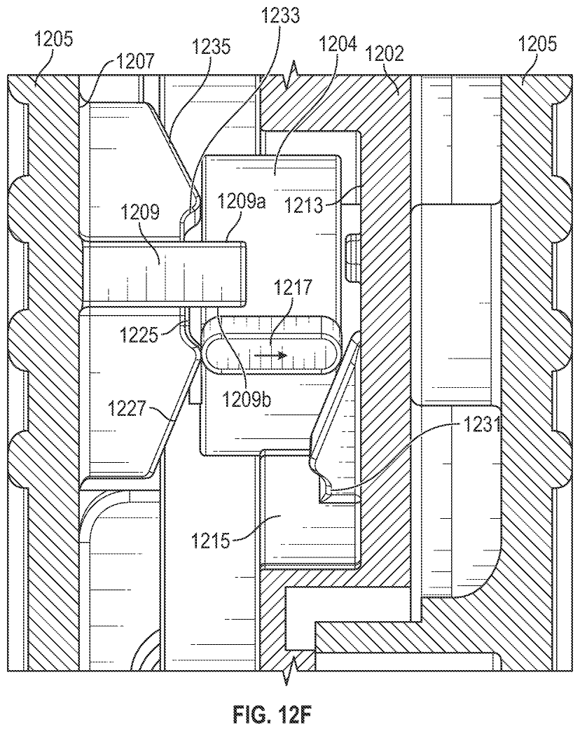

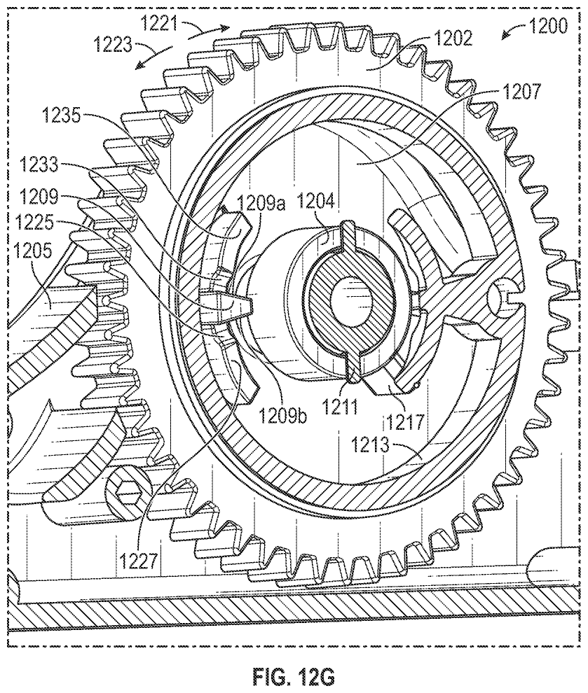

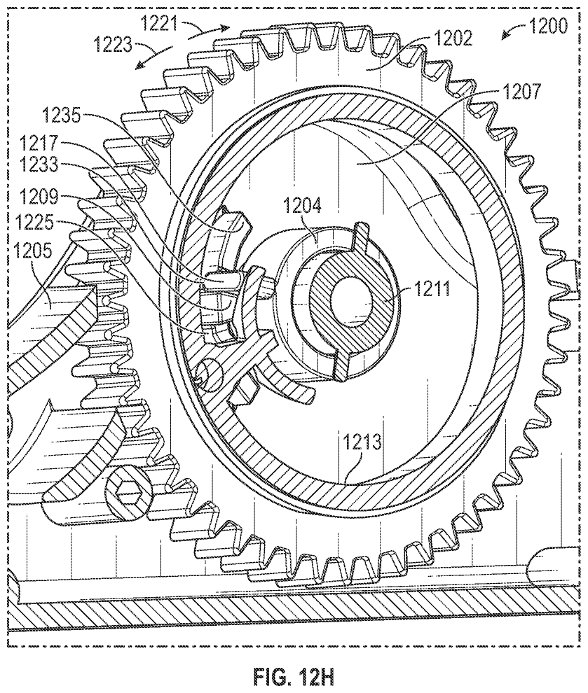

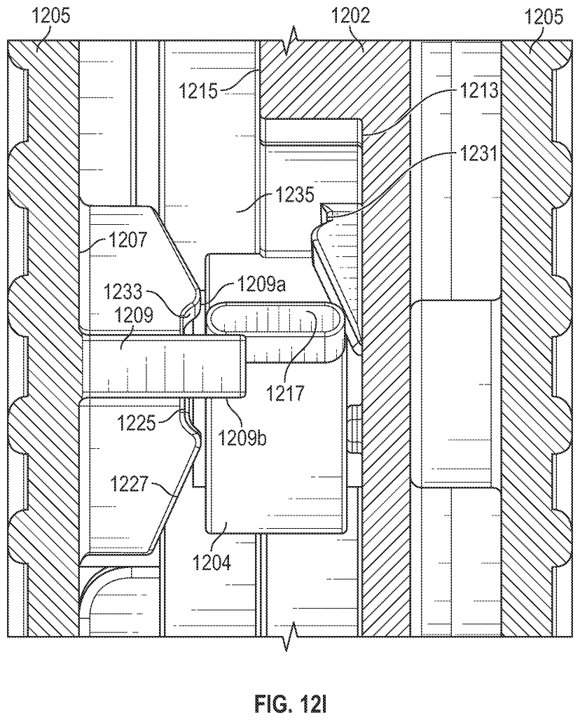

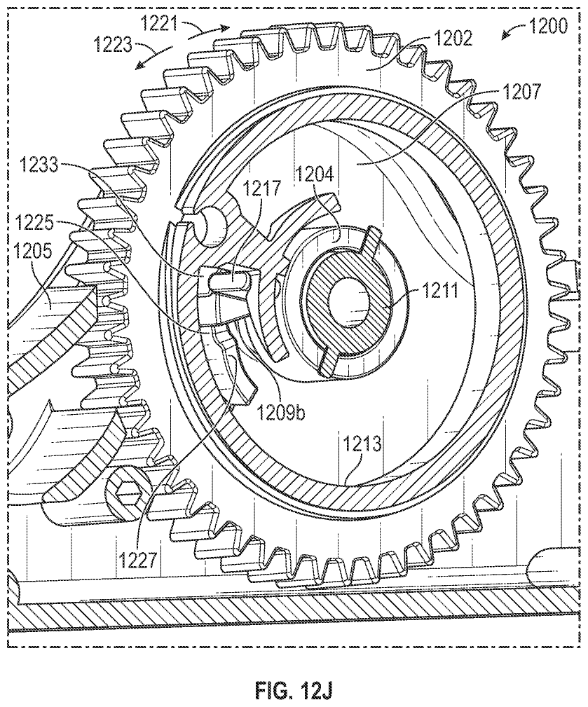

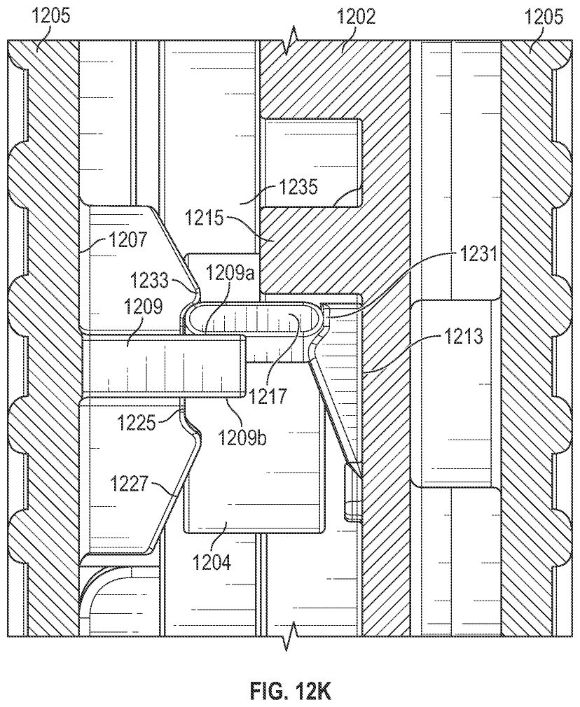

In some embodiments, additional geometry can be added to the first wheel 1102 and/or housing 1105 so there would be a detent at a beginning and/or an end of rotation or travel of the first wheel 1102. In some embodiments, multiple detents may act against the second wheel 1104, and the second wheel 1104 may slide axially to allow one detent to act at a time. The additional geometry may include different ramp angles to encourage one ramp to act before the other. An example the additional geometry is illustrated in FIG. 12.

FIGS. 12A-12K illustrate another example of an instrument delivery device 1200, in accordance with some embodiments. In some embodiments, the instrument delivery device 1200 may be similar or identical to one or more of the following in terms of one or more features and/or operation: the instrument delivery device 100, the instrument delivery device 200, the instrument delivery device 300, the instrument delivery device 400, the instrument delivery device 500, the instrument delivery device 600, the instrument delivery device 700, the instrument delivery device 800, the instrument delivery device 900, the instrument delivery device 1000, and the instrument delivery device 1100. In some embodiments, the instrument delivery device 1200 may be moved from a position of FIGS. 12A/12B to FIG. 12C/12D to FIG. 12E/12F to FIG. 12G to FIG. 12H/12I to FIG. 12J/12K.

In some embodiments, the instrument delivery device 1200 may include a first wheel 1202 and a second wheel 1204. In some embodiments, the first wheel 1202 of the instrument delivery device 1200 may include or correspond to the advancement wheel 152 of FIG. 1, the spool 155 of FIG. 1, the spool 251 of FIG. 2, the spool 351 of FIG. 3, the spool 451 of FIG. 4, the advancement wheel 552 of FIG. 5, the first guide wheel 555 of FIG. 5, the pinion 655 of FIG. 6, the advancement wheel 752 of FIG. 7, the advancement wheel 1052 of FIG. 10, or the first wheel 1102 of FIG. 11.

In some embodiments, the instrument delivery device 1200 may include a housing 1205, which may include a distal end and a proximal end. In some embodiments, the distal end of the housing 1205 may be configured to couple to an intravenous catheter device. In some embodiments, the housing 1205 of the instrument delivery device 1200 may include or correspond to the housing 105 of FIG. 1, the housing 205 of FIG. 2, the housing 305 of FIG. 3, the housing 405 of FIG. 4, the housing 505 of FIG. 5, the housing 605 of FIG. 6, the housing 705 of FIG. 7, the housing 805 of FIG. 8, the housing 1005 of FIG. 10, or the housing 1105 of FIG. 11.

In some embodiments, an inner surface 1207 of the housing 1205 may include a housing stop member 1209, which may include a protrusion. In some embodiments, the housing stop member 1209 may include a first side 1209a and a second side 1209b, which may be opposite the first side 1209a.

In some embodiments, the instrument delivery device 1200 may include an axle 1211, and the first wheel 1202 may be configured to rotate with the axle 1211, which may be concentric with the first wheel 1202. In some embodiments, an inner surface 1213 of the first wheel 1202 may include a first wheel stop member 1215, which may include a protrusion. In some embodiments, a gap may be disposed between the housing stop member 1209 and the first wheel stop member 1215. In some embodiments, the second wheel 1204 may include a tab 1217 configured to bridge the gap between the housing stop member 1209 and the first wheel stop member 1215. In some embodiments, the second wheel 1204 may be disposed on the axle 1211. In some embodiments, the second wheel 1204 may be configured to rotate with the axle 1211 and move axially along the axle 1211.

In some embodiments, the instrument delivery device 1200 may include an instrument. In some embodiments, the instrument delivery device 1200 may include an instrument. In some embodiments, the instrument of the instrument delivery device 1100 may include or correspond to the instrument 153 of FIG. 1, the instrument 253 of FIG. 2, the instrument 353 of FIG. 3, the instrument 453 of FIG. 4, the instrument 553 of FIG. 5, the instrument 653 of FIG. 6, the instrument 753 of FIG. 7, the instrument 853 of FIG. 8, or the instrument 1053 of FIG. 10. In some embodiments, the instrument may include a wire constructed of nickel titanium or another suitable material. In some embodiments, the first wheel 1202 may be configured to rotate to advance the instrument 1219 in a distal direction through the distal end of the housing 1205. In some embodiments, the first wheel 1202 may be configured to rotate more than one full turn.

In some embodiments, the inner surface 1207 of the housing 1205 may include a housing detent 1225. In some embodiments, the inner surface 1207 of the housing 1205 may include a housing ramped surface 1127. In some embodiments, the housing detent 1225 may be disposed between the housing stop member 1209 and the housing ramped surface 1227. In some embodiments, the inner surface 1213 of the first wheel 1202 may include a first wheel detent 1229. In some embodiments, the inner surface 1213 may include a first wheel ramped surface 1231. In some embodiments, the first wheel detent 1229 may be disposed between the first wheel stop member 1215 and the first wheel ramped surface 1231.

In some embodiments, in response to the instrument delivery device 1200 being disposed in a first configuration, the tab 1217 may be disposed within the housing detent 1225 and the first wheel detent 1229 and the first wheel 1202. In these embodiments, the first wheel 1202 may be prevented from rotating in a first direction 1221 but may be configured to rotate in a second direction 1223 opposite the first direction 1221. In some embodiments, the first wheel 1202 may be configured to rotate more than one full turn in the second direction 1223. In some embodiments, the housing detent 1225 may be disposed across from the first wheel detent 1229 in the first configuration.

In some embodiments, the first wheel 1202 may be configured to rotate in the second direction 1223 to advance the instrument in the distal direction through the distal end of the housing 1205. In some embodiments, in response to the first wheel 1202 rotating in the second direction from the first configuration, the tab 1217 may be removed from the first wheel detent 1229 before the tab 1217 is removed from the housing detent 1225. In these embodiments, the tab 1217 may be removed from the housing detent 1225 in response to the tab 1217 sliding toward the inner surface 1213 of the first wheel 1202. In some embodiments, the tab 1217 may be removed from the first wheel detent 1229 before the tab 1217 is removed from the housing detent 1225 because the housing detent 1225 may be shallower than the first wheel detent 1229 or have a shallower angle for the tab 1217 to ramp out of the housing detent 1225.

In some embodiments, the housing ramped surface 1227 may be ramped towards the housing detent 1225. In some embodiments, the first wheel ramped surface 1231 may be ramped towards the first wheel detent 1229. In some embodiments, the housing ramped surface 1227 and the first wheel ramped surface 1231 may be ramped in differing directions.

In some embodiments, the inner surface 1207 of the housing 1205 may include another housing detent 1233 and/or another housing ramped surface 1235. In some embodiments, the other housing detent 1233 may be disposed between the housing stop member 1209 and the other housing ramped surface 1235 and on an opposite side of the housing stop member 1209 as the housing detent 1225. In some embodiments, in response to the instrument delivery device 1200 being disposed in the second configuration, the tab 1217 may be disposed within the other housing detent 1233 and another first wheel detent 1239.

In some embodiments, the first wheel 1202 may be configured to rotate from the first configuration to the second configuration, and the tab 1217 may be disposed within the other housing detent 1233 and the other first wheel detent 1239 in the second configuration. In some embodiments, in order to move from the first configuration to the second configuration, the tab 1217 may contact the housing stop member 1209. In some embodiments, in response to the tab 1217 contacting the housing stop member 1209 and further rotation of the first wheel 1202 in the second direction, the tab 1217 may move along another first wheel ramped surface 1237 and may be pushed by the other first wheel ramped surface 1237 towards the inner surface 1207 of the housing 1205 and into the other housing detent 1233.

In some embodiments, when the tab 1217 may snap into and/or snap out of the housing detent 1225, the other housing detent 1233, and the first wheel detent 1229, which may provide resistance to movement or rotation. In some embodiments, one or more of the housing ramped surface 1227, the first wheel ramped surface 1231, and the other housing ramped surface 1235 may facilitate guidance of the tab 1217 and the second wheel 1204 and may move the second wheel 1204 axially along the axle 1211.

In some embodiments, in response to the first wheel 1202 and the second wheel 1204 moving in the second direction 1223 from the first configuration to the second configuration, the tab 1217 may contact the housing stop member 1209. In some embodiments, in response to the tab 1217 contacting the housing stop member 1209 and further rotation of the first wheel 1202 in the second direction 1223, the tab 1217 may move along other first wheel ramped surface 1237 and is pushed by the other first wheel ramped surface 1237 into the other housing detent 1233. In some embodiments, when the tab 1217 is inserted into the other housing detent 1233, it may snap into other first wheel detent 1239. In some embodiments, the other first wheel ramped surface 1231 may be ramped towards the first wheel detent 1229 and may facilitate guidance of the tab 1217.

In some embodiments, as illustrated in FIG. 12, the inner surface 1207 of the housing 1205 may include the other housing detent 1233. It is understood that in some embodiments, the instrument delivery device 1200 may be modified such that the tab 1217 snaps out of the housing detent 1225 first, prior to snapping out of the first wheel detent 1229. In these embodiments, the first wheel 1202 may include the other housing detent 1233 and/or the other ramped surface 1235, which may be disposed on an opposite side of the first wheel stop member 1215 as the first wheel detent 1229. In these embodiments, a particular arrangement on the inner surface 1207 of one or more of the housing ramped surface 1227, the other ramped surface 1235, the housing stop member 1209, the housing detent 1225, stop member, and the other housing detent 1233 illustrated in FIG. 12 may instead be disposed on the inner surface of the first wheel 1202. In these embodiments, one or more of the first wheel ramped surface 1231, the other first wheel ramped surface 1237, the first wheel stop member 1215, the first wheel detent 1229, and the other first wheel detent 1239 may instead be disposed on the inner surface 1207 of the housing 1205. In some embodiments, in the second configuration, the tab 1217 may be disposed within the other housing detent 1233 on the inner surface 1213 of the first wheel and one of the housing detent and the first wheel detent. In these and other embodiments, the first direction 1221 and the second direction 1223 may be reversed.

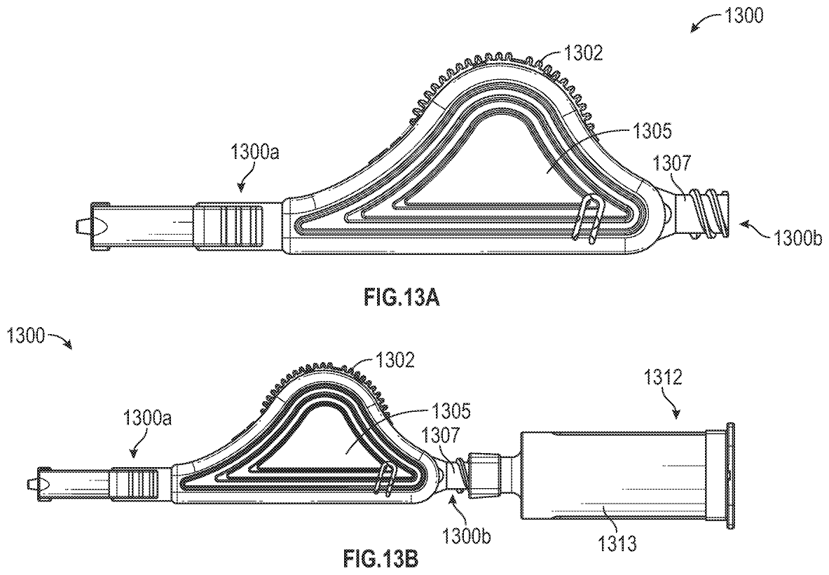

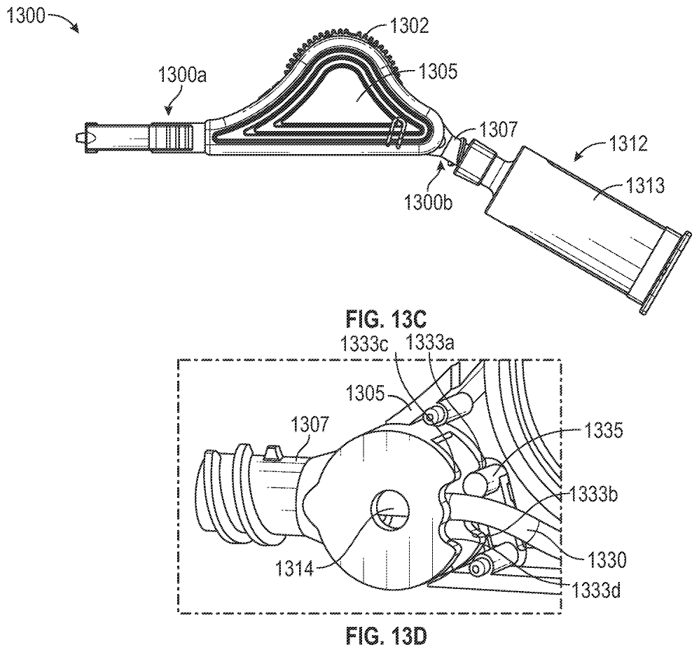

FIGS. 13A-13D illustrate another example of an instrument delivery device 1300. In some embodiments, the instrument delivery device 1300 may be similar or identical to one or more of the following in terms of one or more features and/or operation: the instrument delivery device 100, the instrument delivery device 200, the instrument delivery device 300, the instrument delivery device 400, the instrument delivery device 500, the instrument delivery device 600, the instrument delivery device 700, the instrument delivery device 800, the instrument delivery device 900, the instrument delivery device 1000, the instrument delivery device 1100, and the instrument delivery device 1200.

In some embodiments, an advancement wheel 1302 may be at least partially disposed within a housing 1305 of the instrument delivery device 1300. In some embodiments, the advancement wheel 1302 of the instrument delivery device 1200 may include or correspond to the advancement wheel 152 of FIG. 1, the spool 155 of FIG. 1, the spool 251 of FIG. 2, the spool 351 of FIG. 3, the spool 451 of FIG. 4, the advancement wheel 552 of FIG. 5, the first guide wheel 555 of FIG. 5, the pinion 655 of FIG. 6, the advancement wheel 752 of FIG. 7, the advancement wheel 1052 of FIG. 10, the first wheel 1102 of FIG. 11, or the first wheel 1202 of FIG. 12.

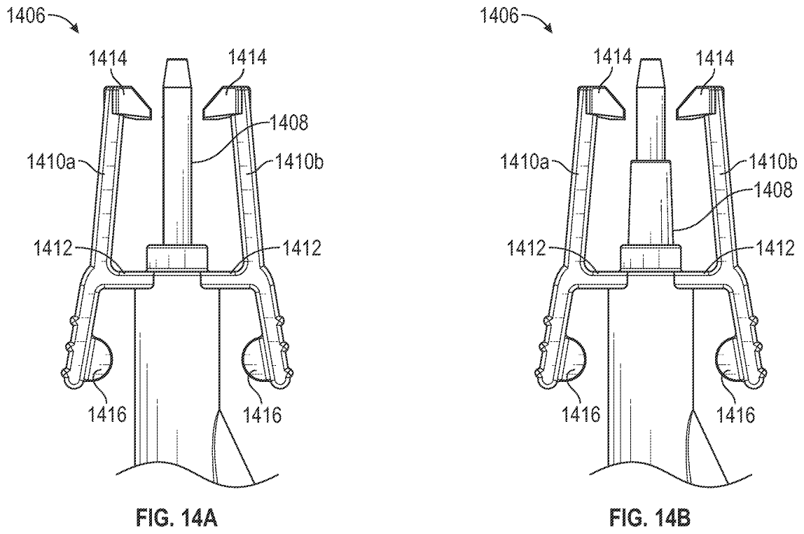

In some embodiments, the instrument delivery device 1300 may include the housing 1305, which may include a distal end 1300a and a proximal end 1300b. In some embodiments, the proximal end 1300b may include or correspond to the proximal end of the housing in one or more of FIGS. 1-12. In some embodiments, the distal end 1300a may include any type of distal connector to configured to couple to the instrument delivery device 1200 to be coupled to an IV catheter device or could incorporate an IV catheter. In some embodiments, the distal end 1300a may include a distal connector 1406, described further with respect to FIGS. 14A-14B.

In some embodiments, the advancement wheel 1302 may extend out of the housing 1305, and in response to the advancement wheel 1302 being rotated, an instrument may be advanced through the distal end 1300a of the housing 1305. In some embodiments, the instrument of the instrument delivery device 1300 may include or correspond to the instrument 153 of FIG. 1, the instrument 253 of FIG. 2, the instrument 353 of FIG. 3, the instrument 453 of FIG. 4, the instrument 553 of FIG. 5, the instrument 653 of FIG. 6, the instrument 753 of FIG. 7, the instrument 853 of FIG. 8, or the instrument 1053 of FIG. 10.

In some embodiments, the proximal end 1300b may include a proximal connector 1307, which may be adjustable. In further detail, in some embodiments, an angle of the proximal connector 1307 may be adjustable with respect to the housing 1305. In some embodiments, the proximal connector 1307 may be adjustable between a first position with respect to the housing 1305 and a second position with respect to the housing 1305. In some embodiments, the proximal connector 1307 may be adjustable to more than two positions with respect to the housing 1305. In some embodiments, the proximal connector 1307 may be disposed at a first angle with respect to the housing 1305 when in the first position and at a second angle with respect to the housing 1305 when in the second position.

In some embodiments, the first position may correspond to a horizontal position with respect to the housing 1305. In some embodiments, the second position may correspond to an upwardly inclined position with respect to the housing 1305 or a downwardly inclined position with respect to the housing 1305, illustrated, for example, in FIG. 13C. In some embodiments, the proximal connector 1307 may be adjustable between the horizontal position and the upwardly inclined position and/or the downwardly inclined position. In some embodiments, when the proximal connector 1307 is in the horizontal position, the proximal connector may be aligned with a longitudinal axis of the instrument delivery device 1300 and/or the distal connector, such as, for example, the distal connector 1406. In some embodiments, when the proximal connector 1307 is in the horizontal position, the proximal connector 1307 may be generally aligned with the ground or with an arm of a patient in which the IV catheter device is inserted.

In some embodiments, the proximal connector 1307 may include a luer connector, such as a female luer connector, or another suitable connector. In some embodiments, a blood collection device 1312 may be coupled to the proximal connector 1307, and a blood sample can be collected through a fluid pathway extending through the housing 1305. In some embodiments, the fluid pathway may extend through a tube 1330 coupled to and/or integrated with the proximal connector 1307 and/or the distal end 1300a. In some embodiments, the blood collection device 1312 may include a sharp cannula surrounded by a holder 1313, which may be configured to receive a container such as a test tube or BD VACUTAINER® Blood Collection Tube, available from Becton Dickinson & Company of Franklin Lakes, N.J.

In some embodiments, the proximal connector 1307 may be pivotally coupled to the housing 1305. In some embodiments, the proximal connector 1307 may pivot around a pivot point 1314. In these and other embodiments, the proximal connector 1307 may be configured to move along an axis between the first position and the second position. In these and other embodiments, movement or adjustment of the proximal connector 1307 may be one-dimensional.

As illustrated in FIG. 13D, in some embodiments, the proximal connector 1307 may include one or more detents 1333 configured to hold the proximal connector 1307 in a particular position or at a particular angle with respect to the housing 1305. In some embodiments, the detents 1333 may facilitate use of the proximal connector 1307 (and the blood collection device 1312 or another device coupled to it) as a handle by the clinician during operation of the instrument delivery device 1300. In some embodiments, the proximal connector 1307 may be used as a handle in the first position and/or the second position. In some embodiments, the proximal connector 1307 may be adjusted from the horizontal position to the upwardly inclined position and/or the downwardly inclined position to improve visualization of blood during blood collection, allow better coating of the blood from additives in the test tube or BD VACUTAINER® Blood Collection Tube, and/or prevent additive mixing if the blood were allowed to flow distally back into the sharp cannula of the blood collection device 1312.

In some embodiments, the proximal connector 1307 may include a first detent 1333a and a second detent 1333b (which may be referred to collectively in the present disclosure as “detents 1333”). In some embodiments, an inner surface of the housing 1305 may include a protrusion 1335, which may be configured to fit within the detents 1333 to secure the proximal connector 1307 in the first position and/or the second position. In some embodiments, in response to the proximal connector 1307 being in the horizontal position, the protrusion 1335 may be disposed within the first detent 1333a. In some embodiments, in response to the proximal connector 1307 being in the downwardly inclined position, the protrusion 1335 may be disposed within the second detent 1333b. In some embodiments, the proximal connector 1307 may include a third detent (not illustrated) above the first detent 1333a, which may receive the protrusion 1335 to secure the proximal connector 1307 in the upwardly inclined position. In some embodiments, a third detent 1333c may be similarly shaped to the first detent 1333a and/or a fourth detent 1333d may be similarly shaped to the second detent 1333b. In some embodiments, the protrusion 1335 may be lengthened and configured to extend through the third detent 1333c and the fourth detent 1333d. Alternatively, in some embodiments, the housing 1305 may include another protrusion opposite the protrusion 1335 configured to extend through the third detent 1333c and the fourth detent 1333d.

In some embodiments, the proximal connector 1307 may be coupled to the housing 1305 via a ball-and-socket joint. In some embodiments, the ball-and-socket joint may allow side to side movement of the proximal connector 1307, in addition to movement along the axis. In some embodiments, the ball-and-socket joint may include one or more detents, similar to the detents 1333 of FIG. 13D, or one or more divots or holes. In some embodiments, the housing 1305 may include a protrusion, similar to the protrusion 1335 of FIG. 13D, configured to insert into the detents, divots, or holes. In some embodiments, the protrusion may press towards a center of a ball of the ball-and-socket joint and/or may hold the proximal connector 1307 in one or more positions, such as, for example, the horizontal position, the upwardly inclining position, and the downwardly inclining position. In some embodiments, adjustment of the proximal connector 1307 via the ball-and-socket joint may facilitate more convenient placement of the test tube or BD VACUTAINER® Blood Collection Tube within the holder 1313.

In some embodiments, the housing 1305 may include an upper flange and/or a lower flange. In some embodiments, the proximal connector 1307 may contact the upper flange when the proximal connector 1307 is in the upwardly inclined position and/or contact the lower flange when the proximal connector 1307 is in the downwardly inclined position, which may facilitate use of the proximal connector 1307 as a handle by the clinician and indicate to the clinician a particular position of the proximal connector 1307.

Referring now to FIGS. 14A-14B, in some embodiments, the distal end 1300a may include a distal connector 1406. In some embodiments, the distal connector 1406 may include blunt cannula 1408 and two lever arms 1410a,b disposed on opposite sides of the blunt cannula 1408. In some embodiments, the blunt cannula 1408 may include a blunt cannula, which may be cylindrical, as illustrated, for example, in FIG. 14A.

In some embodiments, the two lever arms 1410a,b may oppose each other and may each be connected to a body of the distal connector 1406 at a flex point 1412. In some embodiments, a hook member 1414 disposed at a distal end of each of the two lever arms 1410a,b may be configured to be disposed within a groove of the IV catheter device when the two lever arms 1410a,b are in a relaxed position, as illustrated, for example, in FIGS. 14A-14B. In some embodiments, the groove may be annular, or the IV catheter device may include separate grooves.

In some embodiments, a proximal end of each of the two lever arms 1410a,b proximal to the flex point 1412 may be pressed inwardly to bias a distal end of each of the lever arms 1410a,b outwardly and release the hook member 1414 from the groove. In some embodiments, the distal end of each of the two lever arms 1410a,b may automatically return from the biased positioned to the relaxed position in response to the proximal end of each of the two lever arms 1410a,b being released or not pressed inwardly.

In some embodiments, the proximal end of each of the two lever arms 1410a,b may include a stop protrusion 1416, which may prevent pressing the two lever arms 1410a,b far enough to yield or break the lever arms 1410a,b. In some embodiments, the stop protrusion 1416 of each of the two lever arms 1410a,b may be disposed opposite each other on the body of the distal connector 1406 instead of on the two lever arms 1410a,b. In some embodiments, the stop protrusion 1416 may limit a distance the two lever arms 1410a,b can be pressed by providing a hard stop.

As illustrated in FIG. 14B, in some embodiments, the blunt cannula may include a luer shape, which may include a luer. In some embodiments, the luer shape may include an annular stepped surface. In some embodiments, the luer shape may facilitate sealing with devices such as, for example, the BD Q-SYTE™ needle-free connector, available from Becton & Dickinson of Franklin Lakes, N.J. In some embodiments, the luer shape may reduce stress on a septum of the BD SMARTSITE™ needle-free connector (available from Becton Dickinson of Franklin Lakes, N.J.) or a similar device and may prevent the septum from being displaced in response to removal of the blunt cannula.

In some embodiments, one or more of the following may include the distal connector 1406: the instrument delivery device 100, the instrument delivery device 200, the instrument delivery device 300, the instrument delivery device 400, the instrument delivery device 500, the instrument delivery device 600, the instrument delivery device 700, the instrument delivery device 800, the instrument delivery device 900, the instrument delivery device 1000, the instrument delivery device 1100, the instrument delivery device 1200, and the instrument delivery device 1300.

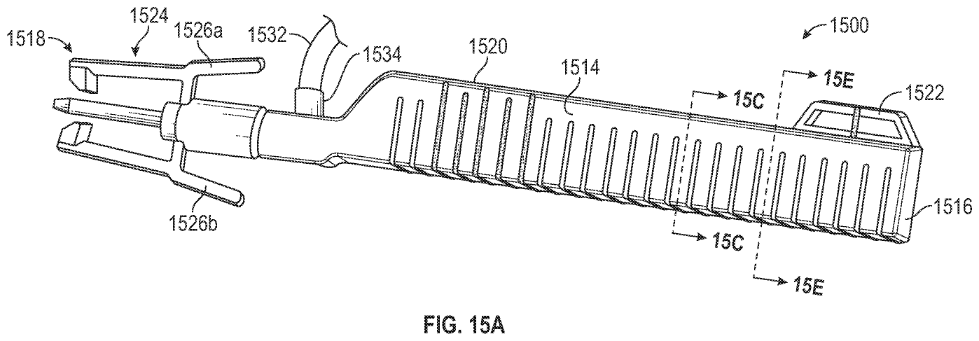

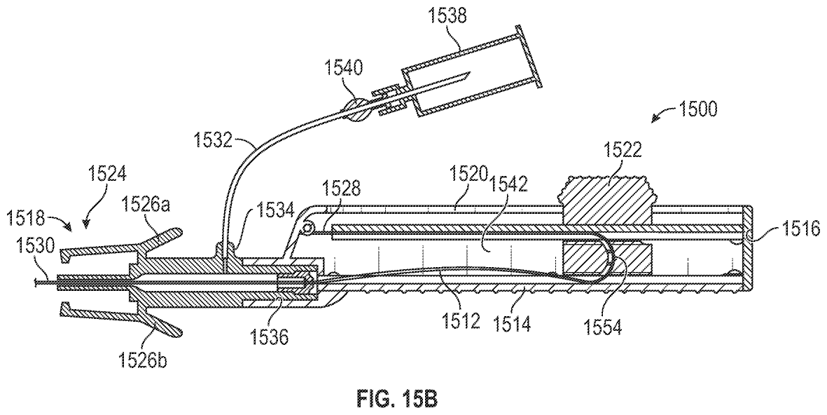

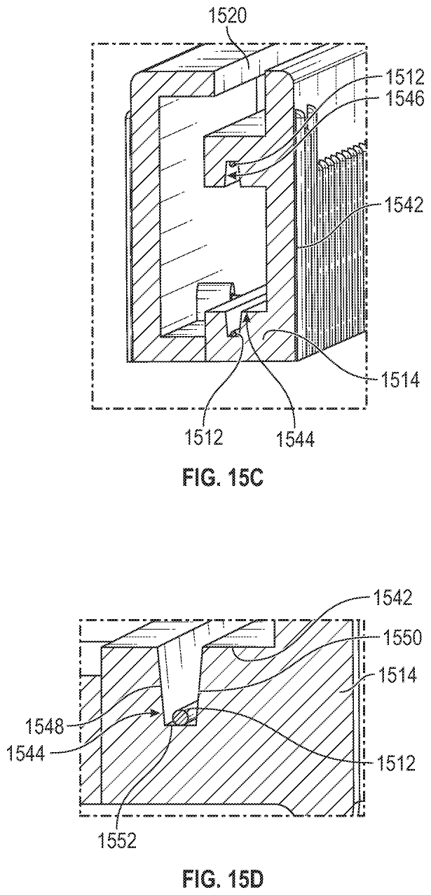

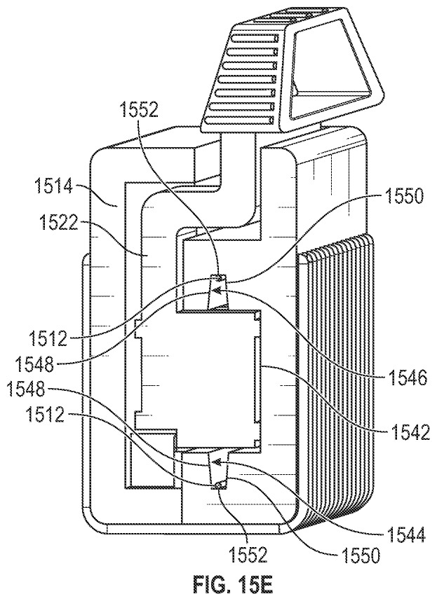

Referring now to FIGS. 15A-15E, in some embodiments, an instrument delivery device 1500 may be configured to deliver an instrument 1512 through a catheter of an IV catheter device. In some embodiments, the instrument 1512 may be advanced through the catheter to push past any occlusions in the catheter or vasculature (e.g., thrombus or fibrin sheath at a tip of the catheter, vein collapse, valves, etc.) to create a clear pathway for fluid flow. In some embodiments, the instrument 1512 may reduce or remove occlusions, improving patency of the catheter for medication and fluid delivery, as well as blood acquisition, during a dwell time of the catheter.

In some embodiments, the instrument delivery device 1300 may be similar or identical to one or more of the following in terms of one or more features and/or operation: the instrument delivery device 100, the instrument delivery device 200, the instrument delivery device 300, the instrument delivery device 400, the instrument delivery device 500, the instrument delivery device 600, the instrument delivery device 700, the instrument delivery device 800, the instrument delivery device 900, the instrument delivery device 1000, the instrument delivery device 1100, the instrument delivery device 1200, and the instrument delivery device 1300.

In some embodiments, the instrument 1512 may include a guidewire, an instrument, a guidewire or an instrument with one or more sensors, or another suitable instrument. In some embodiments, the sensors may be used for patient or device monitoring and may include sensors measuring pressure, temperature, pH, blood chemistry, oxygen saturation, flow rate, or another physiological property. In some embodiments, the instrument 1512 may be similar or identical to the instrument of one or more of FIGS. 1-13 in terms of one or more features and/or operation.

In some embodiments, the catheter may include a peripheral IV catheter, a peripherally-inserted central catheter, or a midline catheter. In some embodiments, the catheter through which the instrument 1512 may be delivered may have been previously inserted into vasculature of a patient and may be dwelling within the vasculature when the instrument 1512 is advanced through the catheter.

In some embodiments, the instrument 1512 may be disposed within a housing 1514, which may be configured to protect the instrument 1512 from damage and/or contamination from a surrounding external environment. In some embodiments, the housing 1514 may be rigid or semi-rigid. In some embodiments, the housing 1514 may be made of one or more of stainless steel, aluminum, polycarbonate, metal, ceramic, plastic, and another suitable material. In some embodiments, the housing 1514 may include a proximal end 1516, a distal end 1518, and a slot 1520. In some embodiments, the slot 1520 may extend parallel to a longitudinal axis of the housing 1514.

In some embodiments, the instrument delivery device 1500 may include an advancement element 1522, which may extend through the slot 1520 and may be configured to move linearly along the slot 1520 between a retracted position illustrated, for example, in FIG. 15A, and an advanced position. In some embodiments, the clinician may pinch or grasp the advancement element 1522 to move the advancement element 1522 between the retracted position and the advanced position.

In some embodiments, the distal end 1518 of the housing 1514 may include a distal connector 1524, which may include or correspond to the distal connector 1406 of FIGS. 14A-14B. In some embodiments, the distal connector 1524 may include opposing lever arms 1526a, 1526b. In some embodiments, distal ends of the opposing lever arms 1526a, 1526b may be configured to move apart from each other in response to pressure applied to proximal ends of the opposing lever arms 1526a,1526b. In some embodiments, in response to removal of the pressure applied to the proximal ends of the opposing lever arms 1526a,1526b, the distal ends may move closer to each other and clasp a portion of the IV catheter device, such as a needleless connector, another connector, or a proximal end of a catheter adapter, for example. In some embodiments, the distal connector 1524 may include a blunt cannula or male luer configured to insert into the portion of the IV catheter device.

In some embodiments, the distal connector 1524 may include any suitable connector. For example, the distal connector 1524 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the distal connector 1524 may include one or more bond pockets, which may each be configured to receive an extension tube. In some embodiments, the distal connector 1524 may be monolithically formed as a single unit with a body of the housing 1514 that includes the slot 1520.

In some embodiments, the instrument 1512 may include a first end 1528 and a second end 1530. In some embodiments, movement of the advancement element 1522 from the retracted position to the advanced position may cause the second end 1530 of the instrument 1512 to be advanced beyond the distal end 1518 of the housing 1514. In some embodiments, moving the advancement element 1522 to the advanced position may introduce the instrument 1512 into the IV catheter device and/or through the catheter. In some embodiments, in response to the instrument 1512 being introduced into the IV catheter device and/or through the catheter, the instrument 1512 may access a fluid pathway of the IV catheter device and/or the vasculature of a patient.

In some embodiments, the catheter of the IV catheter device with significant dwelling time within the vasculature may be susceptible to narrowing, collapse, kinking, blockage by debris (e.g., fibrin or platelet clots), and adhering of a tip of the catheter to the vasculature. Thus, blood withdrawal using the catheter may be difficult. In some embodiments, the instrument 1512 may have a diameter less than a diameter of the catheter of the IV catheter device to provide access to the vasculature of the patient without any additional needle sticks. In some embodiments, the instrument 1512 may clear the pathway for collecting a blood sample. Thus, in some embodiments, the instrument delivery device 1500 may be used for needle-free blood collection and/or fluid infusion.

In some embodiments, an extension tube 1532 may be coupled to the instrument delivery device 1500, and the extension tube 1532 may be used for blood collection and/or fluid infusion. In some embodiments, the extension tube 1532 may extend from a port 1534 of the housing 1514. In some embodiments, a fluid seal 1536 may be within the housing 1514 to enable the instrument 1512 to advance and/or retract while maintaining a closed fluid path. In some embodiments, the instrument 1512 may be configured to extend through the fluid seal 1536. In some embodiments, the fluid seal 1536 may be disposed proximal to the port 1534 and distal to the advancement element 1522 in the advanced position. In some embodiments, the fluid seal 1536 may include silicone, rubber, an elastomer, or another suitable material. In some embodiments, the fluid seal 1536 may include an aperture, slit, or the like to accommodate the instrument 1512 therethrough.

In some embodiments, a proximal end of the extension tube 1532 may be coupled to a blood collection device 1538. For example, the proximal end of the extension tube 1532 may be integrated with a connector 1540, which may be coupled to the blood collection device 1538. In some embodiments, a needleless connector may be disposed between the connector 1540 and the blood collection device 1538. In some embodiments, the connector 1540 and/or the port 1534 may be coupled to an IV line or another fluidic connection.

In some embodiments, an inner surface 1542 of the housing 1514 may include one or more grooves. For example, the inner surface 1542 may include a first groove 1544 and/or a second groove 1546. In some embodiments, the first groove 1544 and/or the second groove 1546 may be disposed within the housing 1514 between the proximal end 1516 and the distal end 1518. In some embodiments, the instrument 1512 may be disposed within the first groove 1544 and/or the second groove 1546. In some embodiments, the first groove 1544 and/or the second groove 1546 may include a support wall 1548, another support wall 1550 opposite the support wall, and a bottom 1552 extending between the support wall 1548 and the other support wall 1550. In some embodiments, the first groove 1544 and/or the second groove 1546 may be open opposite the bottom 1552. In some embodiments, the first groove 1544 and/or the second groove 1546 may be linear and/or configured to guide the instrument 1512 as the instrument 1512 is advanced distally and/or retracted proximally.

In some embodiments, the instrument 1512 may be linear within the housing 1514 and the first end 1528 of the instrument 1512 may be coupled to the advancement element 1522. In other embodiments, the advancement element 1522 may include an arc-shaped channel 1554, which may be U-shaped. In some embodiments, the instrument 1512 may extend and move through the arc-shaped channel 1554. In some embodiments, the first end 1528 of the instrument 1512 may be fixed. In some embodiments, the first end 1528 of the instrument may be fixed within the housing 1514. In some embodiments, in response to movement of the advancement element 1522 a first distance, the second end of the instrument 1512 may be configured to advance distally a second distance. In some embodiments, the second distance may be twice the first distance. In some embodiments, the second distance may be more than twice the first distance. In these and other embodiments, the instrument 1512 may extend through multiple U-shapes or other arc-shapes. In some embodiments, because the first groove 1544 and/or the second groove 1546 are open opposite the bottom 1552, the instrument 1512 may tend to buckle in response to the advancement element 1522 being advanced distally, as illustrated, for example, in FIG. 15B.

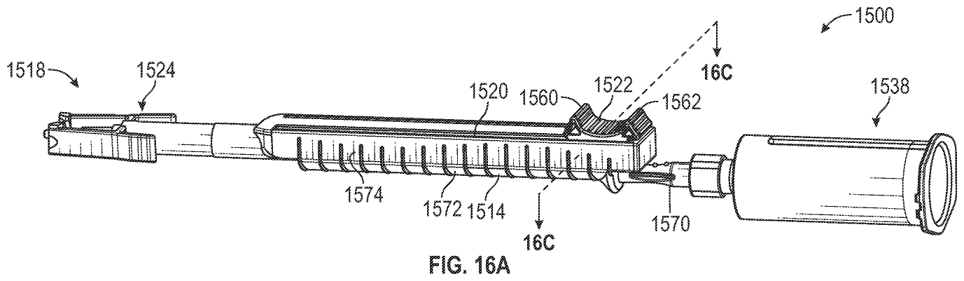

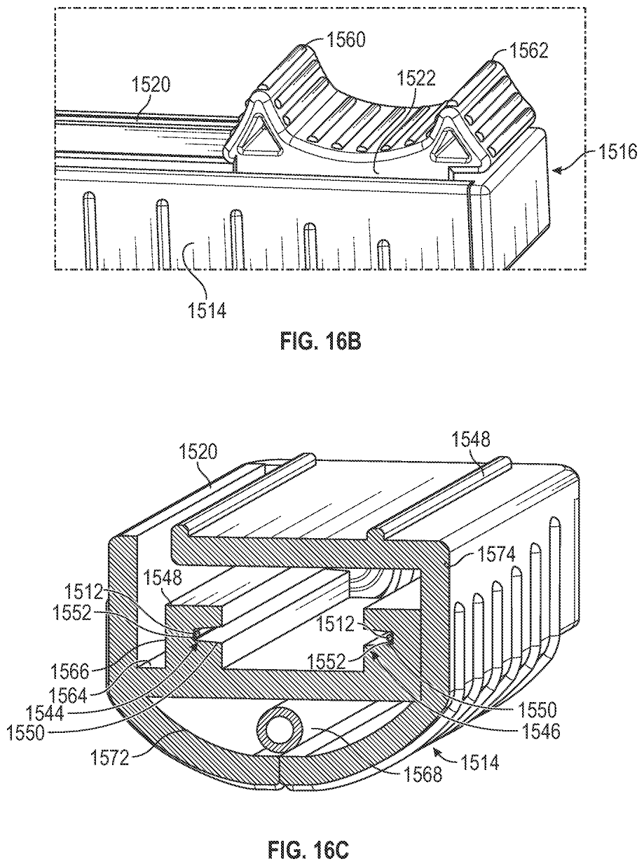

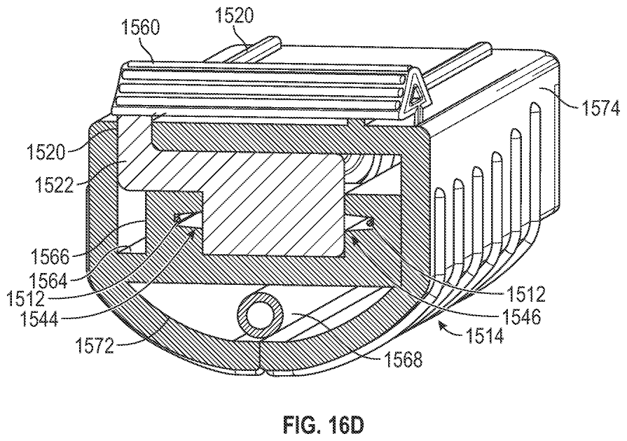

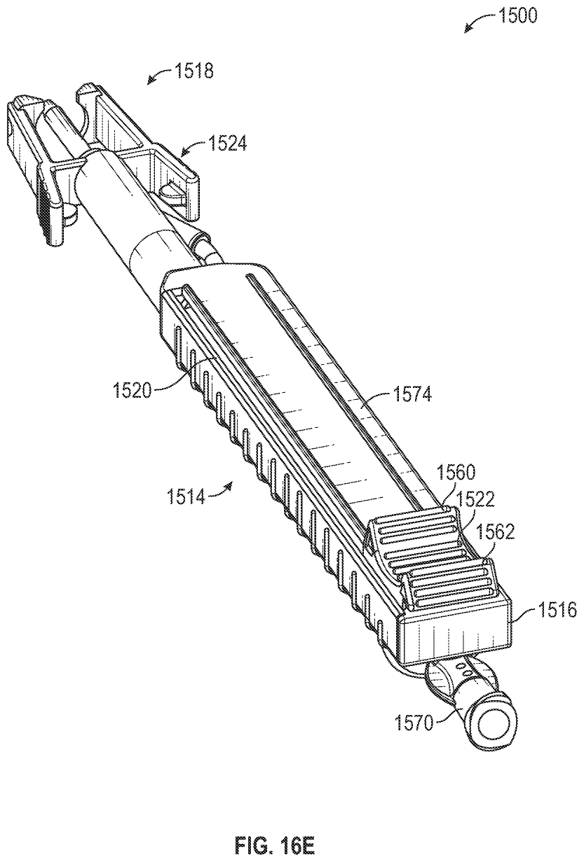

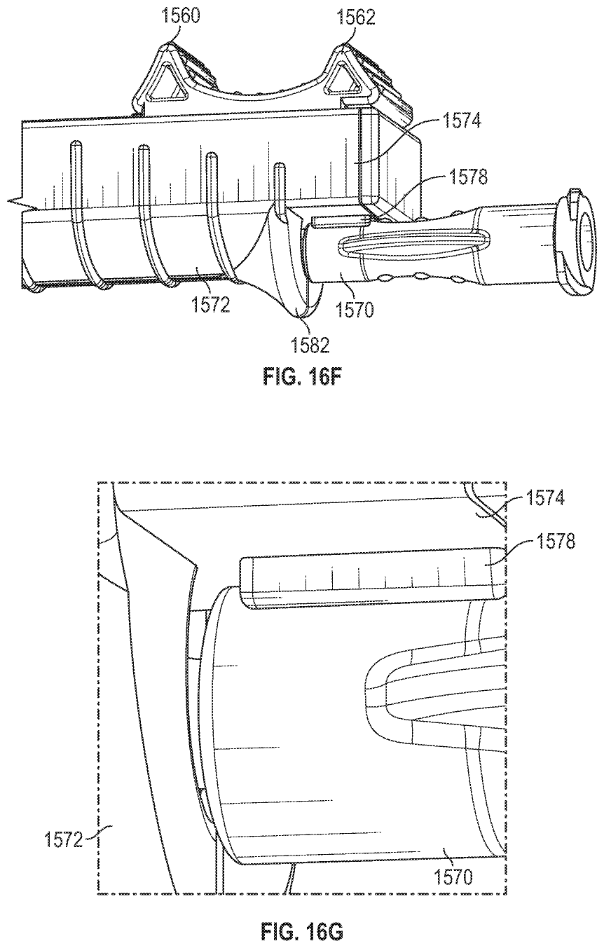

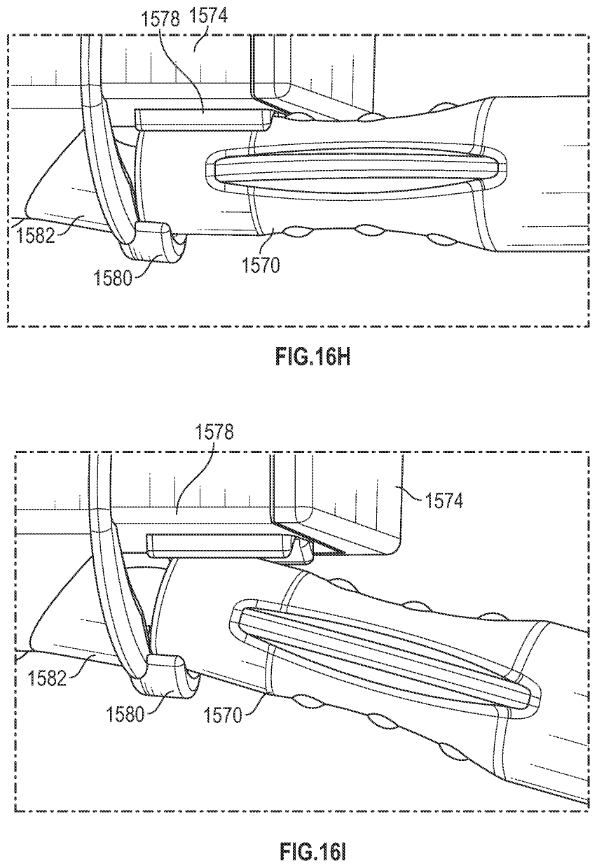

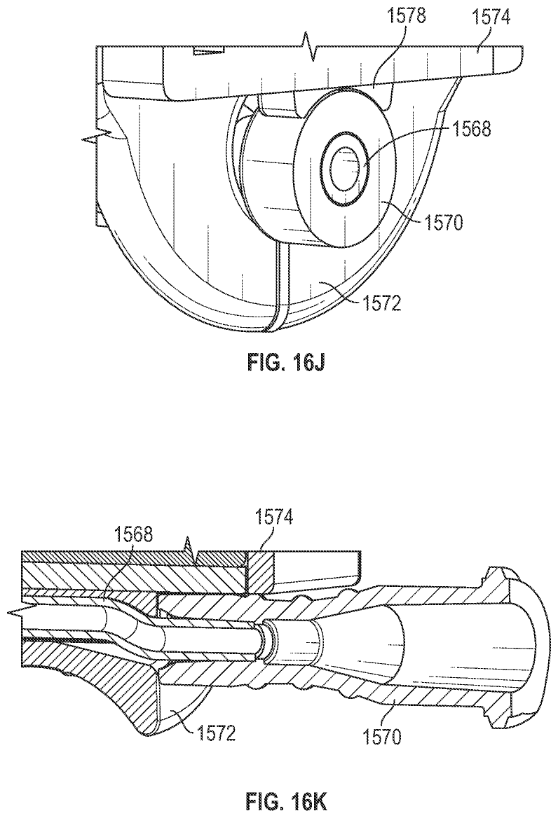

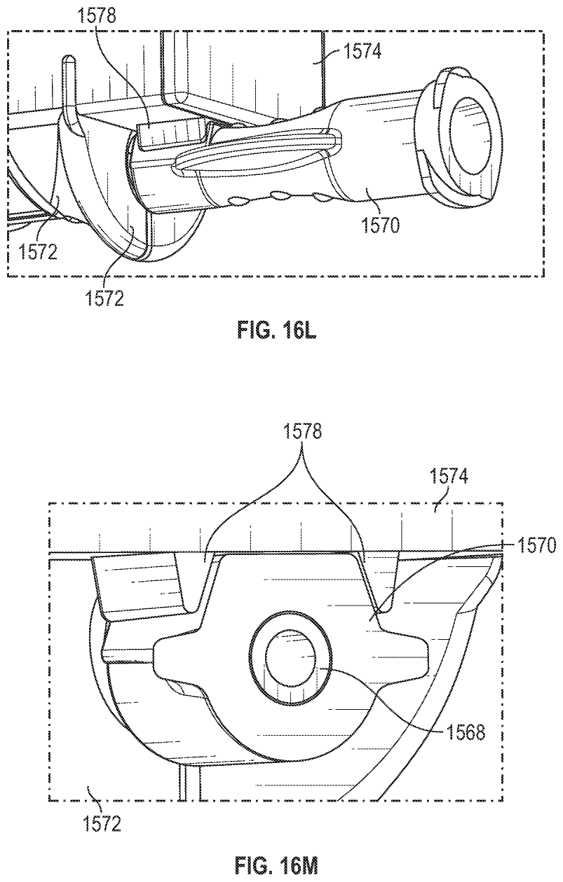

Referring now to FIGS. 16A-16E, the instrument delivery device 1500 is illustrated, according to some embodiments. In some embodiments, the instrument delivery device 1500 may be similar or identical to the instrument delivery device 1500 of FIG. 15 in terms of one or more features and/or operation. In some embodiments, an upper surface of the advancement element 1522 may include a first push tab 1560 and a second push tab 1562, which may allow the clinician to advance the instrument 1512 in a distal direction without repositioning his or her hand. The advancement element 1522 may otherwise need to be slid further in the distal direction than an average hand size can slide the advancement element 1522 in one push, and the clinician would reposition his or her hand to continue to push the advancement element 1522 in the distal direction. In some embodiments, to advance the instrument 1512 from the retracted position to the advanced position, the clinician may reposition his or her finger but not his or her hand grip due to the second push tab 1562 in addition to the first push tab 1560. In some embodiments, the advancement element 1522 may include more than two push tabs, which may extend a length of the advancement element 1522.

In some embodiments, the first push tab 1560 may be distal to the second push tab 1562 and/or at a distal end of the advancement element 1522. In some embodiments, the second push tab 1562 may be at a proximal end of the advancement element 1522. In some embodiments, the first push tab 1560 and the second push tab 1562 may be a same height, which may facilitate securement of the finger of clinician. In some embodiments, the first push tab 1560 and the second push tab 1562 may be different heights. For example, the first push tab 1560 may be taller than the second push tab 1562, which may make the upper surface of the advancement element 1522 easier to push on if the finger cannot fit between the first push tab 1560 and the second push tab 1562.

In some embodiments, the housing 1514 is rotated from FIGS. 15A-15E such that the instrument 1512 is disposed in a horizontal plane instead of a vertical plane. In these and other embodiments, the slot 1520 may be disposed on a top of the housing 1514. In some embodiments, positioning of the instrument 1512 in the horizontal plane may change an aspect ratio of the instrument delivery device 1500 from tall and narrow to short and wide, which may facilitate the instrument delivery device 1500 lying flat in a hand of the clinician and may make the instrument delivery device 1500 easier to grip. In some embodiments, a width of the housing 1514 may be greater than a height of the housing 1514, as illustrated, for example, in FIGS. 16A and 16E. Moreover, in some embodiments, positioning of the instrument 1512 in the horizontal plane may allow the advancement element 1522 to be wider and therefore easier to manipulate. In some embodiments, the advancement element 1522 that is wider may be less prone to binding to the housing 1514 and may provide a smoother action and more tactile feedback when the instrument 1512 is advanced. In some embodiments, the horizontal plane may be generally perpendicular to a vertical plane aligned with the force of gravity.

In some embodiments, the housing 1514 may include a dead well 1564 aligned with the slot 1520 and separated from the first groove 1544 and/or the second groove 1546 by a joiner wall 1566 that may form the bottom 1552 and join the support wall 1548 and the other support wall 1550. In some embodiments, the dead well 1564 may include a pocket where any contaminants coming through the slot 1520 may get caught. In some embodiments, the dead well 1564 may be part of a tortuous path that keeps the instrument 1512 sterile after a package of the probe advancement device 1500 has been opened. In some embodiments, any contaminants coming through the slot 1520 must take multiple turns in the tortuous path to reach the instrument 1512 disposed within the first groove 1544 and the second groove 1546, decreasing a likelihood that the contaminants will reach the instrument 1512.

In some embodiments, an extension tube 1568 may extend from the distal connector 1524 and may bypass the fluid seal 1536, extending through the housing 1514 to a proximal connector 1570, which may be disposed at the proximal end 1516 of the housing 1514 or proximal to the proximal end 1516 of the housing 1514. In some embodiments, the proximal connector 1570 may be similar or identical to the proximal connector 1307 of FIG. 13 in terms of one or more features and/or operation. In some embodiments, the blood collection device 1312 may be coupled to the proximal connector 1525.

In some embodiments, the extension tube 1568 may be disposed in a tunnel 1572 of the housing 1514 to reduce a likelihood that the extension tube 1568 interferes with movement of the instrument 1512. In some embodiments, the extension tube 1568 may be used for blood collection and/or fluid infusion. In some embodiments, the extension tube 1568 may be constructed of a flexible material.

Referring now to FIGS. 16F-16G and 16J, in some embodiments, the instrument delivery device 1500 may provide stability between the housing 1514 and the proximal connector 1570 while still allowing some movement between the housing 1514 and the proximal connector 1570. In some embodiments, the movement between the housing 1514 and the proximal connector 1570 may be limited to one or more directions. In some embodiments, the stability may be provided via mating or contact surfaces between the housing 1514 and the proximal connector 1570 that may limit movement in one or more directions but may allow movement in one or more other directions. In further detail, in some embodiments, the tunnel 1572 may terminate distal to a body 1574 of the housing 1514, which may include the instrument 1512 and the advancement element 1522. Thus, the body 1574 may include a contact surface configured to contact the proximal connector 1570 to prevent the proximal connector 1570 from rotating upward.

In some embodiments, the contact surface of the body 1574 may include an upper flange 1578, which may include one or more protrusions from the body 1574 forming an arch or another shape that corresponds to a shape of the proximal connector 1570 to limit side to side movement of the proximal connector 1570. In some embodiments, the extension tube 1568 may come out of the housing 1514 distal to the proximal end 1516, and the proximal connector 1570 may be disposed underneath the body 1574 such that the body 1574 may act as a stop preventing upward movement of the proximal connector 1570. In some embodiments, a proximal end of the proximal connector 1570 may extend proximal to the proximal end 1516. In some embodiments, movement of the proximal connector 1570 may not be limited by the housing 1514.

In some embodiments, movement of the proximal connector 1570, which may include changing an angle of the proximal connector 1570 with respect to the housing 1514, may stretch the extension tube 1568 in length. Thus, in some embodiments, the extension tube 1568 may act like a spring to pull the proximal connector 1570 straight again, or in a horizontal position generally aligned with a longitudinal axis of the instrument delivery device 1500.

Referring now to FIGS. 16H-16I, in some embodiments, the housing 1514 may include a lower flange 1580 that extends from a lower portion of the housing 1514 and contacts a bottom of a distal end of the proximal connector 1570 to provide support to the proximal connector 1570. As illustrated in FIG. 16I, a length of the lower flange 1580 may be short enough to allow the proximal connector 1570 to move downwardly. In some embodiments, the lower flange 1580 may include an arch or another shape that may correspond to a shape of the proximal connector 1570 to limit side to side movement of the proximal connector 1570. In some embodiments, the housing 1514 may include a hole or a socket that the distal end of the proximal connector 1570 may rest in.

In some embodiments, the upper flange 1578 and/or the lower flange 1580 may stabilize the proximal connector 1570 such that the proximal connector 1570 may be used as a handle by the clinician during operation of the instrument delivery device 1500 or while the instrument 1512 is being advanced and/or retracted. In some embodiments, the upper flange 1578 and/or the lower flange 1580 may allow repositioning of the proximal connector 1570 or changing of the angle of the proximal connector 1570 with respect to the housing 1514. In some embodiments, the proximal connector 1570 may be adjusted from the horizontal position to a downwardly inclined position to improve visualization of blood during blood collection, allow better coating of the blood from additives in the test tube or BD VACUTAINER® Blood Collection Tube, and/or prevent additive mixing if the blood were allowed to flow distally back into the sharp cannula of the blood collection device 1312.

In some embodiments, the proximal connector 1570 may be pivotally connected to the housing 1514 in a same or similar manner as the proximal connector 1307. In further detail, in some embodiments, the proximal connector 1570 may include one or more detents 1333 and the housing 1514 may include the protrusion 1335 (see, for example, FIGS. 13A-13D). In these and other embodiments, the proximal connector 1570 may be configured to move between the horizontal position and the upwardly inclined position and/or between the horizontal position and the downwardly inclined position. In some embodiments, the proximal connector 1570 may be coupled to the housing 1514 via the ball-and-socket joint described with respect to FIG. 13.

In some embodiments, the housing 1514 may include a finger catch 1582 near the proximal end 1516 that may facilitate gripping of the housing 1514 by the clinician. In some embodiments, the finger catch 1582 may be repeated along a bottom of the housing 1514 to make recesses for multiple fingers along the bottom of the housing 1514. In some embodiments, the lower flange 1580 may extend proximally from the finger catch 1582, which may include a bump and/or flange extending outwardly and downwardly from the bottom of the housing 1514.

Referring now to FIG. 16K, the body 1574 may be configured to contact the proximal connector 1570 to prevent the proximal connector 1570 from rotating upward. In some embodiments, the extension tube 1568 may be integrated with the proximal connector 1570.

Referring now to FIGS. 16L-16M, in some embodiments, the upper flange 1578 may be an acute trapezoid, which may be symmetric, and may match a corresponding shape of the proximal connector 1570.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. An instrument delivery device, comprising:
a housing, comprising a distal end and a proximal end, wherein the distal end is configured to couple to an intravenous catheter device, wherein the proximal end comprises a proximal connector, wherein the proximal connector is adjustable between a first position with respect to the housing to a second position with respect to the housing;
an instrument disposed within the housing; and
an advancement wheel, wherein the advancement wheel extends out from the housing, wherein in response to the advancement wheel being rotated, the instrument is advanced through the distal end of the housing.

2. The instrument delivery device of claim 1, wherein the proximal connector comprises a luer connector.

3. The instrument delivery device of claim 1, wherein the proximal connector is pivotally coupled to the housing and configured to move along an axis between the first position and the second position.

4. The instrument delivery device of claim 3, wherein the proximal connector comprises a first detent and a second detent, wherein an inner surface of the housing comprises a protrusion, wherein in response to the proximal connector being in the first position, the protrusion is disposed within the first detent, wherein in response to the proximal connector being in the second position, the protrusion is disposed within the second detent.

5. The instrument delivery device of claim 1, wherein the first position is a horizontal position with respect to the housing, wherein the second position is a downwardly inclined position with respect to the housing.

6. The instrument delivery device of claim 5, wherein the proximal connector comprises a first detent and a second detent, wherein an inner surface of the housing comprises a protrusion, wherein in response to the proximal connector being in the horizontal position, the protrusion is disposed within the first detent, wherein in response to the proximal connector being in the downwardly inclined position, the protrusion is disposed within the second detent.

7. The instrument delivery device of claim 1, wherein the distal end comprises a distal connector, wherein the distal connector comprises a blunt cannula and two lever arms disposed on opposite sides of the blunt cannula, wherein the blunt cannula comprises a luer shape, wherein a proximal end of each of the two lever arms comprises a stop protrusion.

8. An instrument delivery device, comprising:
a housing, comprising a proximal end, a distal end, and a slot, wherein an inner surface of the housing comprises a groove disposed within the housing between the proximal end of the housing and the distal end of the housing;
an advancement element extending through the slot and configured to move linearly along the slot between a retracted position and an advanced position, wherein the advancement element comprises a first push tab and a second push tab; and
an instrument comprising a first end and a second end, wherein in response to movement of the advancement element from the retracted position to the advanced position, the second end of the instrument is advanced beyond the distal end of the housing.

9. The instrument delivery device of claim 8, wherein the advancement element comprises an arc-shaped channel, wherein the instrument extends through the arc-shaped channel, wherein the first end of the instrument is fixed, wherein in response to movement of the advancement element a first distance, the second end of the instrument is configured to advance distally a second distance, wherein the second distance is at least twice the first distance, wherein an inner surface of the housing comprises a first groove and a second groove, wherein the second groove is generally parallel to the first groove, wherein the instrument is disposed within the first groove and the second groove.

10. The instrument delivery device of claim 8, wherein the instrument is oriented in a horizontal plane.

11. The instrument delivery device of claim 10, wherein a width of the housing is greater than a height of the housing.

12. The instrument delivery device of claim 8, wherein the first push tab is disposed at a distal end of the advancement element, wherein the second push tab is disposed at a proximal end of the advancement element and is aligned with the first push tab.

13. The instrument delivery device of claim 12, wherein a height of the first push tab is greater than a height of the second push tab.

14. The instrument delivery device of claim 8, wherein the housing comprises a body and a tunnel disposed underneath the body, further comprising a proximal connector and an extension tube extending through the tunnel and coupled to the proximal connector, wherein the body extends proximal to the tunnel, wherein the body limits upward movement of the proximal connector.

15. The instrument delivery device of claim 14, wherein a bottom surface of the body comprises a protrusion having a corresponding shape to the proximal connector, wherein the protrusion limits upward movement of the proximal connector.

16. The instrument delivery device of claim 15, wherein the protrusion is arched.

17. The instrument delivery device of claim 15, wherein the protrusion is trapezoidal.

18. The instrument delivery device of claim 15, wherein the bottom surface of the body comprises a finger catch proximal to the protrusion.

19. The instrument delivery device of claim 18, wherein the housing further comprises another protrusion extending distally from the finger catch and configured to limit side-to-side movement of the proximal connector.

20. The instrument delivery device of claim 8, wherein the instrument comprises a guidewire.
